SEQUENCE LISTING (172 sequences)

SEQ ID NO: 1 - protein, 142 aa, Homo sapiens
MVVFNGLLKIKICEAVSLKPTAWSLRHAVGPRPQTFLLDPYIALNVDDSRIGQTATKQKTNSPAWHDEFVTDVCNGRKIELAVFHDAPIGYDDFVANCTIQFEELLQNGSRHFEDWIDLEPEGRVYVIIDLSGSSGEAPKDN

SEQ ID NO: 2 - protein, 51 aa, Homo sapiens
PRIKTKRDVNNFDQDFTREEPVLTLVDEAIVKQINQEEFKGFSYFGEDLMP

SEQ ID NO: 3 - protein, 142 aa, Homo sapiens
MAGLGPGVGDSEGGPRPLFCRKGALRQKVVHEVKSHKFTARFFKQPTFCSHCTDFIWGIGKQGLQCQVCSFVVHRRCHEFVTFECPGAGKGPQTDDPRNKHKFRLHSYSSPTFCDHCGSLLYGLVHQGMKCSCCEMNVHRRC

SEQ ID NO: 4 - protein, 65 aa, Homo sapiens
PRPCGRSGENFDKFFTRAAPALTPPDRLVLASIDQADFQGFTYVNPDFVHPDARSPTSPVPVPVM

SEQ ID NO: 5 - protein, 17 aa, Artificial
  synthetic Drosophila antennapedia derived carrier peptide
CRQIKIWFQNRRMKWKK

SEQ ID NO: 6 - protein, 11 aa, Human Immunodeficiency Virus
YGRKKRRQRRR

SEQ ID NO: 7 - protein, 6 aa, Artificial
  synthetic hexa-arginine sequence
RRRRRR

SEQ ID NO: 8 - protein, 7 aa, Artificial
  synthetic hepta-arginine sequence
RRRRRRR

SEQ ID NO: 9 - protein, 10 aa, Artificial
  synthetic deca-arginine sequence
RRRRRRRRRR

SEQ ID NO: 10 - protein, 14 aa, Homo sapiens
KICEAVSLKPTAWS

SEQ ID NO: 11 - protein, 12 aa, Homo sapiens
ICEAVSLKPTAW

SEQ ID NO: 12 - protein, 10 aa, Homo sapiens
CEAVSLKPTA

SEQ ID NO: 13 - protein, 8 aa, Homo sapiens
EAVSLKPT

SEQ ID NO: 14 - protein, 21 aa, Artificial
  synthetic epsilonV1 PKC peptide linked to TAT
EAVSLKPTGGYGRKKRRQRRR

SEQ ID NO: 15 - protein, 13 aa, Homo sapiens
PYIALNVDDSRIG

SEQ ID NO: 16 - protein, 14 aa, Homo sapiens
AVFHDAPIGYDDFV

SEQ ID NO: 17 - protein, 12 aa, Homo sapiens
VFHDAPIGYDDF

SEQ ID NO: 18 - protein, 8 aa, Homo sapiens
HDAPIGYD

SEQ ID NO: 19 - protein, 19 aa, Artificial
  synthetic epsilonV1-7 PKC peptide linked to TAT
HDAPIGYDYGRKKRRQRRR

SEQ ID NO: 20 - protein, 15 aa, Homo sapiens
AVGPRPQTFLLDPYI

SEQ ID NO: 21 - protein, 14 aa, Homo sapiens
DDSRIGQTATKQKT

SEQ ID NO: 22 - protein, 17 aa, Homo sapiens
HDEFVTDVCNGRKIELA

SEQ ID NO: 23 - protein, 13 aa, Homo sapiens
DLSGSSGEAPKDN

SEQ ID NO: 24 - protein, 13 aa, Homo sapiens
PRIKTKRDVNNFD

SEQ ID NO: 25 - protein, 8 aa, Homo sapiens
IKTKRDVN

SEQ ID NO: 26 - protein, 11 aa, Homo sapiens
QDFTREEPVLT

SEQ ID NO: 27 - protein, 11 aa, Homo sapiens
LVDEAIVKQIN

SEQ ID NO: 28 - protein, 16 aa, Homo sapiens
QEEFKGFSYFGEDLMP

SEQ ID NO: 29 - protein, 12 aa, Homo sapiens
MAGLGPGVGDSE

SEQ ID NO: 30 - protein, 14 aa, Homo sapiens
GGPRPLFCRKGALR

SEQ ID NO: 31 - protein, 16 aa, Homo sapiens
QKVVHEVKSHKFTARF

SEQ ID NO: 32 - protein, 18 aa, Homo sapiens
FKQPTFCSHCTDFIWGIG

SEQ ID NO: 33 - protein, 10 aa, Homo sapiens
KQGLQCQVCS

SEQ ID NO: 34 - protein, 12 aa, Homo sapiens
FVVHRRCHEFVT

SEQ ID NO: 35 - protein, 18 aa, Homo sapiens
FECPGAGKGPQTDDPRNK

SEQ ID NO: 36 - protein, 14 aa, Homo sapiens
HKFRLHSYSSPTFC

SEQ ID NO: 37 - protein, 16 aa, Homo sapiens
DHCGSLLYGLVHQGMK

SEQ ID NO: 38 - protein, 12 aa, Homo sapiens
CSCCEMNVHRRC

SEQ ID NO: 39 - protein, 12 aa, Homo sapiens
PRPCGRSGENFD

SEQ ID NO: 40 - protein, 7 aa, Homo sapiens
CGRSGEN

SEQ ID NO: 41 - protein, 11 aa, Homo sapiens
GENFDKFFTRA

SEQ ID NO: 42 - protein, 14 aa, Homo sapiens
TPPDRLVLASIDQA

SEQ ID NO: 43 - protein, 6 aa, Homo sapiens
RLVLAS

SEQ ID NO: 44 - protein, 13 aa, Homo sapiens
IDQADFQGFTYVN

SEQ ID NO: 45 - protein, 15 aa, Homo sapiens
PDFVHPDARSPTSPV

SEQ ID NO: 46 - protein, 142 aa, Rattus norvegicus
MVVFNGLLKIKICEAVSLKPTAWSLRHAVGPRPQTFLLDPYIALNVDDSRIGQTATKQKTNSPAWHDEFVTDVCNGRKIELAVFHDAPIGYDDFVANCTIQFEELLQNGSRHFEDWIDLEPEGKVYVIIDLSGSSGEAPKDN

SEQ ID NO: 47 - protein, 51 aa, Rattus norvegicus
PRIKTKRDVNNFDQDFTREEPILTLVDEAIVKQINQEEFKGFSYFGEDLMP

SEQ ID NO: 48 - protein, 142 aa, Rattus norvegicus
MAGLGPGGGDSEGGPRPLFCRKGALRQKVVHEVKSHKFTARFFKQPTFCSHCTDFIWGIGKQGLQCQVCSFVVHRRCHEFVTFECPGAGKGPQTDDPRNKHKFRLHSYSSPTFCDHCGSLLYGLVHQGMKCSCCEMNVHRRC

SEQ ID NO: 49 - protein, 19 aa, Artificial
  synthetic construct of gammaV5 PKC peptide linked to TAT
RLVLASGGYGRKKRRQRRR

SEQ ID NO: 50 - protein, 10 aa, Homo sapiens
SFNSYELGSL

SEQ ID NO: 51 - protein, 23 aa, Artificial
  synthetic construct of deltaV1-1 PKC peptide linked to TATthrough a disulfide bridge
CSFNSYELGSLCYGRKKRRQRRR

SEQ ID NO: 52 - protein, 6 aa, Homo sapiens
FDLGSL

SEQ ID NO: 53 - protein, 6 aa, Homo sapiens
YDIGSL

SEQ ID NO: 54 - protein, 6 aa, Homo sapiens
YDVGSL

SEQ ID NO: 55 - protein, 6 aa, Homo sapiens
YDLPSL

SEQ ID NO: 56 - protein, 6 aa, Homo sapiens
YDLGLL

SEQ ID NO: 57 - protein, 6 aa, Homo sapiens
YDLGSI

SEQ ID NO: 58 - protein, 6 aa, Homo sapiens
YDLGSV

SEQ ID NO: 59 - protein, 4 aa, Homo sapiens
IGSL

SEQ ID NO: 60 - protein, 4 aa, Homo sapiens
VGSL

SEQ ID NO: 61 - protein, 4 aa, Homo sapiens
LPSL

SEQ ID NO: 62 - protein, 4 aa, Homo sapiens
LGLL

SEQ ID NO: 63 - protein, 4 aa, Homo sapiens
LGSI

SEQ ID NO: 64 - protein, 4 aa, Homo sapiens
LGSV

SEQ ID NO: 65 - protein, 11 aa, Homo sapiens
ALSTERGKTLV

SEQ ID NO: 66 - protein, 11 aa, Homo sapiens
ALSTDRGKTLV

SEQ ID NO: 67 - protein, 11 aa, Homo sapiens
ALTSDRGKTLV

SEQ ID NO: 68 - protein, 11 aa, Homo sapiens
ALTTDRGKSLV

SEQ ID NO: 69 - protein, 11 aa, Homo sapiens
ALTTDRPKTLV

SEQ ID NO: 70 - protein, 11 aa, Homo sapiens
ALTTDRGRTLV

SEQ ID NO: 71 - protein, 11 aa, Homo sapiens
ALTTDKGKTLV

SEQ ID NO: 72 - protein, 10 aa, Homo sapiens
ALTTDKGKTL

SEQ ID NO: 73 - protein, 10 aa, Homo sapiens
VLMRAAEEPV

SEQ ID NO: 74 - protein, 10 aa, Homo sapiens
QSMRSEDEAK

SEQ ID NO: 75 - protein, 9 aa, Homo sapiens
AFNSYELGS

SEQ ID NO: 76 - protein, 9 aa, Homo sapiens
QGFEKKTGV

SEQ ID NO: 77 - protein, 9 aa, Homo sapiens
DNNGTYGKI

SEQ ID NO: 78 - protein, 6 aa, Homo sapiens
KNLIDS

SEQ ID NO: 79 - protein, 8 aa, Homo sapiens
VKSPRDYS

SEQ ID NO: 80 - protein, 9 aa, Homo sapiens
VKSPCRDYS

SEQ ID NO: 81 - protein, 8 aa, Homo sapiens
IKSPRLYS

SEQ ID NO: 82 - protein, 6 aa, Homo sapiens
KNLIDS

SEQ ID NO: 83 - protein, 11 aa, Homo sapiens
PKVKSPRDYSN

SEQ ID NO: 84 - protein, 7 aa, Homo sapiens
NGLLKIK

SEQ ID NO: 85 - protein, 11 aa, Homo sapiens
LAVFHDAPIGY

SEQ ID NO: 86 - protein, 9 aa, Homo sapiens
DDFVANCTI

SEQ ID NO: 87 - protein, 10 aa, Homo sapiens
WIDLEPEGRV

SEQ ID NO: 88 - protein, 10 aa, Homo sapiens
HAVGPRPQTF

SEQ ID NO: 89 - protein, 8 aa, Homo sapiens
NGSRHFED

SEQ ID NO: 90 - protein, 8 aa, Homo sapiens
HDAPIGDY

SEQ ID NO: 91 - protein, 6 aa, Homo sapiens
HDAPIG

SEQ ID NO: 92 - protein, 8 aa, Homo sapiens
HDAAIGYD

SEQ ID NO: 93 - protein, 8 aa, Homo sapiens
HDAPIPYD

SEQ ID NO: 94 - protein, 8 aa, Homo sapiens
HNAPIGYD

SEQ ID NO: 95 - protein, 8 aa, Homo sapiens
HAAPIGYD

SEQ ID NO: 96 - protein, 8 aa, Homo sapiens
ADAPIGYD

SEQ ID NO: 97 - protein, 8 aa, Homo sapiens
HDAPAGYD

SEQ ID NO: 98 - protein, 8 aa, Homo sapiens
HDAPIGAD

SEQ ID NO: 99 - protein, 8 aa, Homo sapiens
HDAPIAYD

SEQ ID NO: 100 - protein, 8 aa, Homo sapiens
HDAPIGYA

SEQ ID NO: 101 - protein, 9 aa, Homo sapiens
SSPSEEDRS

SEQ ID NO: 102 - protein, 8 aa, Homo sapiens
PCDQEIKE

SEQ ID NO: 103 - protein, 9 aa, Homo sapiens
ENNIRKALS

SEQ ID NO: 104 - protein, 8 aa, Homo sapiens
GEVRQGQA

SEQ ID NO: 105 - protein, 6 aa, Homo sapiens
EAIVKQ

SEQ ID NO: 106 - protein, 7 aa, Homo sapiens
IKTKRDV

SEQ ID NO: 107 - protein, 7 aa, Homo sapiens
IKTKRLI

SEQ ID NO: 108 - protein, 7 aa, Homo sapiens
CEAIVKQ

SEQ ID NO: 109 - protein, 10 aa, Homo sapiens
TKRDVNNFDQ

SEQ ID NO: 110 - protein, 7 aa, Homo sapiens
VRLKAHY

SEQ ID NO: 111 - protein, 6 aa, Homo sapiens
VDSEGD

SEQ ID NO: 112 - protein, 8 aa, Homo sapiens
VFPSIPEQ

SEQ ID NO: 113 - protein, 15 aa, Homo sapiens
SQEPPVDDKNEDADL

SEQ ID NO: 114 - protein, 7 aa, Homo sapiens
IKDDSED

SEQ ID NO: 115 - protein, 9 aa, Homo sapiens
PVIDGMDGI

SEQ ID NO: 116 - protein, 6 aa, Homo sapiens
EDAIKR

SEQ ID NO: 117 - protein, 5 aa, Homo sapiens
EDAIR

SEQ ID NO: 118 - protein, 8 aa, Homo sapiens
ITDDYGLD

SEQ ID NO: 119 - protein, 8 aa, Homo sapiens
ITDDYGDL

SEQ ID NO: 120 - protein, 7 aa, Homo sapiens
DDYGLDN

SEQ ID NO: 121 - protein, 7 aa, Homo sapiens
NGYLRVR

SEQ ID NO: 122 - protein, 8 aa, Homo sapiens
EAVGLQPT

SEQ ID NO: 123 - protein, 11 aa, Homo sapiens
LAVFHETPLGY

SEQ ID NO: 124 - protein, 8 aa, Homo sapiens
DFVANCTL

SEQ ID NO: 125 - protein, 10 aa, Homo sapiens
WVDLEPEGKV

SEQ ID NO: 126 - protein, 8 aa, Homo sapiens
HSLFKKGH

SEQ ID NO: 127 - protein, 9 aa, Homo sapiens
TGASDTFEG

SEQ ID NO: 128 - protein, 6 aa, Homo sapiens
EGHLPM

SEQ ID NO: 129 - protein, 6 aa, Homo sapiens
EGHDPM

SEQ ID NO: 130 - protein, 8 aa, Homo sapiens
IKSREDVS

SEQ ID NO: 131 - protein, 8 aa, Homo sapiens
VRSREDVS

SEQ ID NO: 132 - protein, 9 aa, Homo sapiens
PRIKSREDV

SEQ ID NO: 133 - protein, 10 aa, Homo sapiens
HQVRVKAYYR

SEQ ID NO: 134 - protein, 11 aa, Homo sapiens
YELNKDSELLI

SEQ ID NO: 135 - protein, 15 aa, Homo sapiens
MDQSSMHSDHAQTVI

SEQ ID NO: 136 - protein, 7 aa, Homo sapiens
LDQVGEE

SEQ ID NO: 137 - protein, 9 aa, Homo sapiens
EAMNTRESG

SEQ ID NO: 138 - protein, 6 aa, Homo sapiens
DDIVRK

SEQ ID NO: 139 - protein, 8 aa, Homo sapiens
VKLCDFGF

SEQ ID NO: 140 - protein, 8 aa, Homo sapiens
IRLCDFAF

SEQ ID NO: 141 - protein, 10 aa, Homo sapiens
QVKLCDFGFA

SEQ ID NO: 142 - protein, 9 aa, Homo sapiens
MSVPPLLRP

SEQ ID NO: 143 - protein, 11 aa, Homo sapiens
KFPECGFYGLY

SEQ ID NO: 144 - protein, 9 aa, Homo sapiens
DPDADQEDS

SEQ ID NO: 145 - protein, 9 aa, Homo sapiens
SKDTLRKRH

SEQ ID NO: 146 - protein, 9 aa, Homo sapiens
ITLFQNDTG

SEQ ID NO: 147 - protein, 9 aa, Homo sapiens
GSNSHKDIS

SEQ ID NO: 148 - protein, 6 aa, Homo sapiens
SDSPEA

SEQ ID NO: 149 - protein, 8 aa, Homo sapiens
GLSNFDCG

SEQ ID NO: 150 - protein, 11 aa, Homo sapiens
YVESENGQMYI

SEQ ID NO: 151 - protein, 10 aa, Homo sapiens
IVKGKNVDLI

SEQ ID NO: 152 - protein, 10 aa, Homo sapiens
DMNEFETEGF

SEQ ID NO: 153 - protein, 9 aa, Homo sapiens
CSIKNEARL

SEQ ID NO: 154 - protein, 9 aa, Homo sapiens
GKREPQGIS

SEQ ID NO: 155 - protein, 9 aa, Homo sapiens
DEVDKMCHL

SEQ ID NO: 156 - protein, 6 aa, Homo sapiens
RALINS

SEQ ID NO: 157 - protein, 8 aa, Homo sapiens
VKSPFDCS

SEQ ID NO: 158 - protein, 8 aa, Homo sapiens
VRSPFDCS

SEQ ID NO: 159 - protein, 7 aa, Homo sapiens
DRALINS

SEQ ID NO: 160 - protein, 8 aa, Homo sapiens
ISGEFGLD

SEQ ID NO: 161 - protein, 8 aa, Homo sapiens
CSGEFGLD

SEQ ID NO: 162 - protein, 7 aa, Homo sapiens
DDDIVRK

SEQ ID NO: 163 - protein, 6 aa, Homo sapiens
KLFIMN

SEQ ID NO: 164 - protein, 6 aa, Homo sapiens
QEVIRN

SEQ ID NO: 165 - protein, 8 aa, Homo sapiens
VPPEGSEA

SEQ ID NO: 166 - protein, 6 aa, Homo sapiens
QLVIAN

SEQ ID NO: 167 - protein, 14 aa, Human Immunodeficiency Virus
GRKKRRQRRRPPQC

SEQ ID NO: 168 - protein, 19 aa, Human Immunodeficiency Virus
LGISYGRKKRRQRRRPPQC

SEQ ID NO: 169 - protein, 24 aa, Human Immunodeficiency Virus
FITKALGISYGRKKRRQRRRPPQC

SEQ ID NO: 170 - protein, 17 aa, Human Immunodeficiency Virus
FITKALGISYGRKKRRC

SEQ ID NO: 171 - protein, 160 aa, Homo sapiens
MAPFLRISFNSYELGSLQAEDDASQPFCAVKMKEALTTDRGKTLVQKKPTMYPEWKSTFDAHIYEGRVIQIVLMRAAEDPMSEVTVGVSVLAERCKKNNGKAEFWLDLQPQAKVLMCVQYFLEDGDCKQSMRSEEEAMFPTMNRRGAIKQAKIHYIKNHE

SEQ ID NO: 172 - protein, 55 aa, Homo sapiens
PKVKSPSDYSNFDPEFLNEKPQLSFSDKNLIDSMDQEAFHGFSFVNPKFEQFLDI

(19) United States
(12) Patent Application Publication
(10) Pub. No.: US 2010/0311671 A1
(43) Pub. Date: Dec. 9, 2010

(54) TRANSDERMAL DELIVERY OF PKC MODULATORY PEPTIDES THROUGH MICROPORATED SKIN

(75) Inventors: Randolph Mellus Johnson, Half Moon Bay, CA (US); Felix Karim, Walnut Creek, CA (US); Lisa Christine Ryner, Pacifica, CA (US)

Correspondence Address: King & Spalding LLP, P.O. Box 889, Belmont, CA, 94002-0889, US

(73) Assignee: Kai Pharmaceuticals, South San Francisco, CA (US)

(21) Appl. No.: 12/732,091

(22) Filed: Mar. 25, 2010

Related U.S. Application Data
(60) Provisional application No. 61/163,403, filed on Mar. 25, 2009.

(51) Int. Cl. A61K 38/16; A61K 38/02; A61K 38/10; A61K 38/08

(52) U.S. Cl. 514/21.2; 514/1.1; 514/21.3; 514/21.4; 514/21.5; 514/21.6; 514/21.7; 514/21.8

(57) ABSTRACT

Disclosed herein are methods for transdermal delivery of PKC modulatory peptides. Generally, methods comprise the delivery of an isozyme specific PKC peptide modulator through skin that has been microporated, e.g., with an array of microneedles. Such methods may be used to administer therapeutically effective amounts of an isozyme selective PKC peptide inhibitor or activator.

### CROSS-REFERENCE TO RELATED APPLICATIONS

This application claims the benefit of U.S. provisional application Ser. No. 61/163,403, filed Mar. 25, 2009, incorporated herein in its entirety by reference.

### REFERENCE TO SEQUENCE LISTING, TABLE OR COMPUTER PROGRAM

A Sequence Listing is being submitted electronically via EFS in the form of a text file, created Mar. 25, 2010 and named “632008002US00seqlist.txt” (42147 bytes), the contents of which are incorporated herein by reference in their entirety.

### TECHNICAL FIELD

Described herein are methods that relate generally to transdermal drug delivery of isozyme specific protein kinase C peptide modulators, and in particular to the transdermal delivery of conjugates comprised of an isozyme specific protein kinase C peptide conjugated to a cell penetrating peptide.

### BACKGROUND

Protein kinase C (“PKC”) is a multigene family of phospholipid-dependent, serine-threonine kinases central to many signal transduction pathways. Molecular cloning studies have identified ten members of the PKC family. These family members, called isozymes, are encoded by nine different genes. The ten isozymes are designated as the α, βI, βII, γ, δ, ε, ζ, η, l/λ and θ isozymes (Y. Nishizuka, Science 258, 607-614 (1992); L. A. Selbie, C. Schmitz-Peiffer, Y. Sheng, T. J. Biden, J. Biol. Chem. 268, 24296-24302 (1993)). Based on sequence homology and biochemical properties, the PKC gene family has been divided into three groups. Members of the classical or cPKC subfamily, α, βI, βII and γPKC, contain four homologous domains (C1, C2, C3 and C4) inter-spaced with isozyme-unique (variable or V) regions, and require calcium and diacylglycerol for activation. Members of the classical PKC family are found in the superficial laminae of the dorsal horn in the spinal cord as well as in numerous brain regions. Members of the novel or nPKC subfamily, δ, ε, η, and θ PKC, lack the C2 homologous domain and do not require calcium for activation. PKCε is found in primary afferent neuron terminals that innervate the spinal cord as well as in numerous brain regions. Finally, members of the atypical or aPKC subfamily, ζ and l/λIPKC, lack both the C2 and one half of the C1 homologous domains and are insensitive to diacylglycerol and calcium. In addition, two related phospholipid-dependent kinases, PKCμ and protein kinase D, share sequence homology in their regulatory domains to novel PKCs and may constitute a new subgroup (F.-J. Johannes, J. Prestle, S. Eis, P. Oberhagemann, K. Pfizenmaier, Biol. Chem. 269, 6140-6148 (1994); A. M. Valverde, J. Sinnett-Smith, J. Van Lint, E. Rozengurt, Proc. Natl. Acad. Sci. USA 91, 8572-8576 (1994)).

It is well established that PKC family proteins play central roles in cell growth and differentiation. PKCs mediate the effects of peptide hormones, growth factors, neurotransmitters and tumor promoters by acting as secondary (downstream, intracellular) messengers for these signaling molecules (Y. Nishizuka, Science 233, 305-312 (1986); Y. Takai, K. Kaibuchi, T. Tsuda, M. Hoshijima, J. Cell. Biochem. 29, 143-155 (1985)). The identities of the PKC isozymes that transduce particular signals in specific cell types are still being determined. The α, βI, βII, γ, δ, ε and ζ isozymes have been implicated in the differentiation of normeural cells (E. Berra, et al., Cell 74, 555-563 (1993); J. Goodnight, H. Mischak, J. F. Mushinski, Adv. Cancer Res. 64, 159-209 (1994); J. R. Gruber, S. Ohno, R. M. Niles, J. Biol. Chem. 267, 13356-13360 (1992); D. E. Macfarlane, L. Manzel, J. Biol. Chem. 269, 4327-4331 (1994); C. T. Powell et al., Proc. Natl. Acad. Sci. USA 89, 147-151 (1992)). Recent studies, showing that the ε isozyme of PKC (“PKCε”) is activated by nerve growth factor (“NGF”) and mediates NGF-induced neurite outgrowth, were interpreted as indicating a role for PKCε in neuronal differentiation (B. Hundle, et al., J. Biol. Chem. 272, 15028-15035 (1997)).

Studies on the subcellular distribution of PKC isozymes demonstrate that activation of PKC results in its redistribution in the cells (also termed translocation), such that activated PKC isozymes associate with the plasma membrane, cytoskeletal elements, nuclei, and other subcellular compartments (Saito, N. et al., Proc. Natl. Acad. Sci. USA, 86:3409 3413 (1989); Papadopoulos, V. and Hall, P. F. J. Cell Biol., 108:553 567 (1989); Mochly-Rosen, D., et al., Molec. Biol. Cell (formerly Cell Reg.), 1:693 706, (1990)). The unique cellular functions of different PKC isozymes are determined by their subcellular location. For example, activated βIPKC is found inside the nucleus, whereas activated βIIPKC is found at the perinucleus and cell periphery of cardiac myocytes (Disatnik, M. H., et al., Exp. Cell Res., 210:287 297 (1994)). The localization of different PKC isozymes to different areas of the cell in turn appears due to binding of the activated isozymes to specific anchoring molecules termed Receptors for Activated C-Kinase (RACKs). RACKs are thought to function by selectively anchoring activated PKC isozymes to their respective subcellular sites. RACKs bind only fully activated PKC and are not necessarily substrates of the enzyme. Nor is the binding to RACKs mediated via the catalytic domain of the kinase (Mochly-Rosen, D., et al., Proc. Natl. Acad. Sci. USA, 88:3997 4000 (1991)). Translocation of PKC reflects binding of the activated enzyme to RACKs anchored to the cell particulate fraction and the binding to RACKs is required for PKC to produce its cellular responses (Mochly-Rosen, D., et al., Science, 268:247 251 (1995)). Inhibition of PKC binding to RACKs in vivo inhibits PKC translocation and PKC-mediated function (Johnson, J. A., et al., J. Biol. Chem., 271:24962 24966 (1996a); Ron, D., et al., Proc. Natl. Acad. Sci. USA, 92:492 496 (1995); Smith, B. L. and Mochly-Rosen, D., Biochem. Biophys. Res. Commun., 188:1235 1240 (1992)).

In general, translocation of PKC is required for proper function of PKC isozymes. Peptides that mimic either the PKC-binding site on RACKs (Mochly-Rosen, D., et al., J. Biol. Chem., 226:1466 1468 (1991a); Mochly-Rosen, D., et al., supra, 1995) or the RACK-binding site on PKC (Ron, et al., supra, 1995; Johnson, J. A., et al., supra, 1996a) are isozyme-specific translocation inhibitors of PKC that selectively inhibit the function of the enzyme in vivo. For example, an eight amino acid peptide derived from εPKC (peptide εV1-2; SEQ ID NO: 13) is described in U.S. Pat. No. 6,165,977. The peptide contains a part of the RACK-binding site on the εPKC and selectively inhibits specific εPKC-mediated functions in cardiac myocytes. Compounds (typically peptides derived from the PKC isoform itself) that that bind to PKC and make it more available for RACK binding selectively activate the function of the enzyme in vivo. Regions of homology between the PKC signaling peptide and its RACK are termed “pseudo-RACK” sequences (ψ-RACK; Ron et al., Proc. Natl. Acad. Sci. USA 91:839-843 (1994); Ron et al., Biol. Chem. 279:24180-24187 (1995)) and typically have a sequence similar to the PKC-binding region of the corresponding RACK. For example, ψεRACK (SEQ ID NO:18) is a ψ-RACK sequence that acts as an εPKC specific agonist and induces translocation of εPKC. ψεRACK administered prior to, during and after exposure to an ishcmic condition reduces the extent of ischemic injury, as described in U.S. Pat. No. 7,081,444.

Individual isozymes of PKC have been implicated in the mechanisms of various disease states, including the following: cancer (α, β, and δ PKC); cardiac hypertrophy and heart failure (βI and βII PKC) nociception (γ and ε PKC); ischemia including myocardial infarction (δ PKC); immune response, particularly T-cell mediated (θ PKC); and fibroblast growth and memory (ζ PKC). Various PKC isozyme- and variable region-specific peptides have been previously described (see, for example, U.S. Pat. No. 5,783,405). The role of εPKC in pain perception has recently been reported (WO 00/01415; U.S. Pat. No. 6,376,467) including therapeutic use of the εV1-2 peptide (a selective inhibitor of εPKC described in the above-referenced '405 patent). The binding specificity for RACK1, a selective anchoring protein for βIIPKC, has recently been reported to reside (at least in part) in the V5 region of βIIPKC (Stebbins, E. et al., J. Biol. Chem. 271:29644-29650 (2001)), including the testing of certain N-, middle, and C-terminus peptides alone, in combination and together with a mixture of three peptides from the εC2 domain. Generally, inhibition of the individual isozyme of PKC, e.g., via peptide inhibitors, or activation of certain individual isozymes of PKC, e.g., via peptide activators, results in the treatment of disease states for which the individual isozyme have been implicated.

Transdermal drug delivery to the body is a desirable and convenient method for delivering biologically active substances to a subject, and in particular for delivery of substances that have poor oral bioavailability, such as proteins and peptides. The transdermal route of delivery has been particularly successful with small (e.g., less than about 1,000 Daltons) lipophilic compounds, such as scopolamine and nicotine, that can penetrate the stratum corneum outer layer of the skin, which serves as an effective barrier to entry of substances into the body. Below the stratum corneum is the viable epidermis, which contains no blood vessels, but has some nerves. Deeper still is the dermis, which contains blood vessels, lymphatics and nerves. Drugs that cross the stratum corneum barrier can generally diffuse to the capillaries in the dermis for absorption and systemic distribution.

Technological advances in transdermal delivery have focused on addressing the need in the art to deliver hydrophilic, high molecular weight compounds, such as proteins and peptides, across the skin. One approach involves disruption of the stratum corneum using chemical or physical methods to reduce the barrier posed by the stratum corneum. Skin microporation technology, which involves the creation of micron dimension transport pathways in the skin using a minimally invasive technique, is a more recent approach. Techniques to create micropores in the skin include thermal microporation or ablation, microneedle arrays, phonophoresis, laser ablation and radiofrequency ablation (Prausnitz and Langer (2008) Nat. Biotechnology 11:1261-68; Arora et al., Int. J. Pharmaceutics, 364:227 (2008); Nanda et al., Current Drug Delivery, 3:233 (2006); Meidan et al. American J. Therapeutics, 11:312 (2004)).

As noted above, inhibition or activation of a selected protein kinase C isozyme involves modulation of an intracellular process whereby the PKC isozyme is translocated to an anchoring site in the cytoplasm or the nucleus of the cell. Delivery of a peptide or protein to inhibit or activate a PKC isozyme thus requires entry of the peptide or protein into the cell. Transdermal delivery of peptides or proteins for modulation of a PKC isozyme must achieve delivery across the stratum corneum followed by delivery across a cell membrane. To date, the art has not demonstrated whether a peptide or protein modulator of a PKC isozyme can be delivered transdermally in an amount sufficient for therapy, and in particular in an amount sufficient for the treatment of a condition.

### BRIEF SUMMARY

In one aspect, a method for transdermally administering a compound is described. In some embodiments, the compound is attached to a cell-penetrating peptide that facilitates transport of the compound across a cell membrane. Application of the compound-carrier protein conjugate to microporated skin achieves delivery of the conjugate locally, systemically, or both.

In another aspect, a method for transdermally administering PKC peptide modulators is described. The method comprises application to microporated skin, a peptide having isozyme specific activity to modulate a PKC isozyme. In a preferred embodiment, the peptide is administered in the form of a conjugate, where the peptide is attached to a cell-penetrating peptide that facilitates transport of the peptide across a cell membrane. Application of the peptide inhibitor-carrier protein conjugate to microporated skin achieves delivery of the conjugate locally, systemically, or both.

In one embodiment, administering comprises application of the conjugate to skin microporated prior to application of the conjugate. In another embodiment, administering comprises application of the conjugate to skin microporated after application of the conjugate. In another embodiment, administering comprises application of the conjugate to skin microporated simultaneous with application of the conjugate.

In another embodiment, administering comprises application of the conjugate to skin microporated by a technique selected from a microneedle array applied to the skin, thermal ablation, laser ablation, ultrasound, or electroporation.

In yet another embodiment, administering comprises application of a microneedle array to the skin, and wherein the conjugate is disposed on an interior or an exterior surface of microneedles in the microneedle array.

In still another embodiment, the method further comprises occluding the microporated skin after application of the conjugate.

In another embodiment, the method comprises administering the conjugate to microporated skin, where the conjugate is in the form of a formulation contained within a transdermal device which is affixed to the microporated skin, or wherein the conjugate is formulated for direct topical application to the skin as a cream, lotion, gel, ointment or the like.

The carrier peptide, in various embodiments, is selected from the group consisting of Antennapedia homeodomain-derived carrier peptide, a Transactivating Regulatory Protein (Tat)-derived transport polypeptide from the Human Immunodeficiency Virus, and a polyarginine.

The PKC modulatory peptide, in one embodiment, has a sequence that has 80% sequence identity with 6-20 contiguous amino acid residues from SEQ ID NO: 1 or SEQ ID NO: 2.

The PKC modulatory peptide, in other embodiments, has a sequence that has 80% sequence identity with 6-20 contiguous amino acid residues from SEQ ID NO: 3 or SEQ ID NO: 4.

In other embodiments, the PKC modulatory peptide has a sequence that has 80% sequence identity with 6-20 contiguous amino acid residues from SEQ ID NO: 171 or SEQ ID NO: 172.

In still other embodiments, the PKC modulatory peptide or the conjugate is modified with an N-terminal or C-terminal chemical moiety.

In one embodiment, the PKC modulatory peptide has isozyme selective activity for γPKC or εPKC, and is administered to a patient experiencing acute pain, chronic pain, neuropathic pain or inflammatory pain.

In another aspect, a method for treating a condition responsive to a selected PKC modulatory peptide is provided. The method comprises contacting microporated skin with a therapeutic conjugate peptide, the conjugate peptide comprised of a PKC modulatory peptide having isozyme selective activity for a PKC attached to a carrier peptide.

In certain embodiments, the selected PKC modulatory peptide is a delta-PKC modulatory peptide, for treating or preventing tissue damage due to ischemia in a patient.

In another embodiment, the peptide inhibitor is covalently attached to a carrier protein, to form a peptide-carrier protein conjugate. The conjugate is applied to the microporated skin, for systemic or local delivery of the peptide in the form of the conjugate. In one embodiment, the conjugate has a sequence identified herein as SEQ ID NO: 14, SEQ ID NO:19, or SEQ ID NO: 51.

In one embodiment, the skin is microporated with a technique selected from a microneedle array, phonophoresis, thermal ablation, laser ablation and radiofrequency ablation. In another embodiment, the skin is microporated using a microporating device comprising an array of microneedles, wherein the microneedles are solid or hollow.

Additional embodiments of the present methods, compositions, and the like will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present methods and compositions. Additional aspects and advantages are set forth in the following description and claims, particularly when considered in conjunction with the accompanying examples and drawings.

### BRIEF DESCRIPTION OF THE DRAWINGS

FIG. 1A is a graph showing the amount, in pg, of an εPKC inhibitor peptide-TAT carrier peptide conjugate (SEQ ID NO:14) in 30 μm planar skin sections, after application of the inhibitor peptide-carrier peptide conjugate to microporated rat skin (diamonds) or intact skin (squares);

FIG. 1B is a graph showing the amount, in pg/mL, of an inhibitor peptide-TAT carrier peptide conjugate (SEQ ID NO:14) as a function of time, in minutes, after application of the inhibitor peptide-carrier peptide conjugate to microporated skin on rats (n=2, squares, diamonds) or to intact skin on rats (n=2, triangles, x symbols);

FIG. 2 is a graph showing the amount, in pg/mL, of an εPKC activator peptide-TAT carrier peptide conjugate (SEQ ID NO:19) as a function of time, in minutes, after application of the activator peptide-carrier peptide conjugate to microporated skin on a rat; and

FIG. 3 is a graph showing the amount, in ng/mL, of a γV5 peptide-TAT carrier peptide conjugate (SEQ ID NO: 49) as a function of time, in hours, after application of the peptide-carrier conjugate to microporated skin on a rat.

### BRIEF DESCRIPTION OF THE SEQUENCES

| PEPTIDE | SEQ ID NO. | SEQUENCE |
|---|---|---|
| εV1 PKC domain, human, | SEQ ID NO: 1 | MVVFNGLLKI KICEAVSLKP |
| amino acids 1-142 of NCBI | | TAWSLRHAVG PRPQTFLLDP |
| Reference Sequence: | | YIALNVDDSR IGQTATKQKT |
| NP_005391.1 | | NSPAWHDEFV TDVCNGRKIE |
| | | LAVFHDAPIG YDDFVANCTI |
| | | QFEELLQNGS RHFEDWIDLE |
| | | PEGRVYVIID LSGSSGEAPK DN |
| εV5 PKC domain, human, | SEQ ID NO: 2 | PRIKTKRDV NNFDQDFTRE |
| amino acids 687-737 of NCBI | | EPVLTLVDEA IVKQINQEEF |
| Reference Sequence: | | KGFSYFGEDL MP |
| NP_005391.1 | | |
| γV1 PKC domain, human, amino | SEQ ID NO: 3 | MAGLGPGVGD SEGGPRPLFC |
| acids 1-142 of NCBI | | RKGALRQKVV HEVKSHKFTA |
| ACCESSION NP_002730 | | RFFKQPTFCS HCTDFIWGIG |
| | | KQGLQCQVCS FVVHRRCHEF |
| | | VTFECPGAGK GPQTDDPRNK |
| | | HKFRLHSYSS PTFCDHCGSL |
| | | LYGLVHQGMK CSCCEMNVHR RC |
| γV5 PKC domain, human, amino | SEQ ID NO: 4 | PRPCGRSGEN FDKFFTRAAP |
| acids 633-697 of NCBI | | ALTPPDRLVL ASIDQADFQG |
| ACCESSION NP_002730 | | FTYVNPDFVH PDARSPTSPV PVPVM |
| Drosophila Antennapedia | SEQ ID NO: 5 | CRQIKIWFQNRRMKWKK |
| homeodomain-derived carrier | | |
| peptide | | |
| HIV TAT Transregulatory Domain | SEQ ID NO: 6 | YGRKKRRQRRR |
| (47-57) | | |
| Hexa-arginine | SEQ ID NO: 7 | RRRRRR |
| Hepta-arginine | SEQ ID NO: 8 | RRRRRRR |
| Deca-arginine | SEQ ID NO: 9 | RRRRRRRRRR |
| εV1 PKC peptide | SEQ ID NO: 10 | KICEAVSLKPTAWS |
| εV1 PKC peptide | SEQ ID NO: 11 | ICEAVSLKPTAW |
| εV1 PKC peptide | SEQ ID NO: 12 | CEAVSLKPTA |
| εV1-2 peptide | SEQ ID NO: 13 | EAVSLKPT |
| εV1 PKC peptide (end capped | SEQ ID NO: 14 | Ac-EAVSLKPT GG YGRKKRRQRRR-NH₂ |
| stabilized) linked to TAT | | |
| εV1 PKC peptide | SEQ ID NO: 15 | PYIALNVDDSRIG |
| εV1 PKC peptide | SEQ ID NO: 16 | AVFHDAPIGYDDFV |
| εV1 PKC peptide | SEQ ID NO: 17 | VFHDAPIGYDDF |
| εV1-7 PKC peptide | SEQ ID NO: 18 | HDAPIGYD |
| εV1-7 PKC peptide (end capped | SEQ ID NO: 19 | A_{C}-HDAPIGYD-AH_{X}-YGRKKRRQRRR-NH₂ |
| stabilized) linked to TAT | | AH_{X} = 6-AMINO-1-HEXANOIC ACID |
| εV1 PKC peptide | SEQ ID NO: 20 | AVGPRPQTFLLDPYI |
| εV1 PKC peptide | SEQ ID NO: 21 | DDSRIGQTATKQKT |
| εV1 PKC peptide | SEQ ID NO: 22 | HDEFVTDVCNGRKIELA |
| εV1 PKC peptide | SEQ ID NO: 23 | DLSGSSGEAPKDN |
| εV5 PKC | SEQ ID NO: 24 | PRIKTKRDVNNFD |
| εV5 PKC | SEQ ID NO: 25 | IKTKRDVN |
| εV5 PKC | SEQ ID NO: 26 | QDFTREEPVLT |
| εV5 PKC | SEQ ID NO: 27 | LVDEAIVKQIN |
| εV5 PKC | SEQ ID NO: 28 | QEEFKGFSYFGEDLMP |
| γV1 PKC | SEQ ID NO: 29 | MAGLGPGVGDSE |
| γV1 PKC | SEQ ID NO: 30 | GGPRPLFC RKGALR |
| γV1 PKC | SEQ ID NO: 31 | QKVV HEVKSHKFTA RF |
| γV1 PKC | SEQ ID NO: 32 | FKQPTFCSHCTDFIWGIG |
| γV1 PKC | SEQ ID NO: 33 | KQGLQCQVCS |
| γV1 PKC | SEQ ID NO: 34 | FVVHRRCHEFVT |
| γV1 PKC | SEQ ID NO: 35 | FECPGAGKGPQTDDPRNK |
| γV1 PKC | SEQ ID NO: 36 | HKFRLHSYSSPTFC |
| γV1 PKC | SEQ ID NO: 37 | DHCGSLLYGLVHQGMK |
| γV1 PKC | SEQ ID NO: 38 | CSCCEMNVHRRC |
| γV5 PKC | SEQ ID NO: 39 | PRPCGRSGENFD |
| γV5 PKC | SEQ ID NO: 40 | CGRSGEN |
| γV5 PKC | SEQ ID NO: 41 | GENFDKFFTRA |
| γV5 PKC | SEQ ID NO: 42 | TPPDRLVLASIDQA |
| γV5 PKC | SEQ ID NO: 43 | RLVLAS |
| γV5 PKC | SEQ ID NO: 44 | IDQADFQGFTYVN |
| γV5 PKC | SEQ ID NO: 45 | PDFVHPDARSPTSPV |
| εV1 PKC domain, rat, | SEQ ID NO: 46 | MVVFNGLLKI KICEAVSLKP |
| amino acids 1-142 of NCBI | | TAWSLRHAVG PRPQTFLLDP |
| Reference Sequence: | | YIALNVDDSR IGQTATKQKT |
| NP_058867.1 | | NSPAWHDEFV TDVCNGRKIE |
| | | LAVFHDAPIG YDDFVANCTI |
| | | QFEELLQNGS RHFEDWIDLE |
| | | PEGKVYVIID LSGSSGEAPK DN |
| εV5 PKC domain, rat, | SEQ ID NO: 47 | PRIK TKRDVNNFDQ DFTREEPILT |
| amino acids 687-737 of NCBI | | LVDEAIVKQI NQEEFKGFSY FGEDLMP |
| Reference Sequence: | | |
| NP_058867.1 | | |
| γV1 PKC domain, rat, amino | SEQ ID NO: 48 | MAGLGPGGGD SEGGPRPLFC |
| acids 1-142 of NCBI Reference | | RKGALRQKVV HEVKSHKFTA |
| Sequence NP_036760 | | RFFKQPTFCS HCTDFIWGIG |
| | | KQGLQCQVCS FVVHRRCHEF |
| | | VTFECPGAGK GPQTDDPRNK |
| | | HKFRLHSYSS PTFCDHCGSL |
| | | LYGLVHQGMK CSCCEMNVHR RC |
| γV5 PKC peptide (end capped | SEQ ID NO: 49 | Ac-RLVLAS GG YGRKKRRQRRR-NH₂ |
| stabilized) linked to TAT | | |
| δV1-1 | SEQ ID NO: 50 | SFNSYELGSL |
| δV1-1 PKC peptide (end capped stabilized) linked to TAT through a disulfide bridge | SEQ ID NO: 51 | 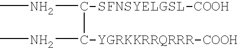 |
| δV1-1.18 | SEQ ID NO: 52 | FDLGSL |
| δV1-1.19 | SEQ ID NO: 53 | YDIGSL |
| δV1-1.20 | SEQ ID NO: 54 | YDVGSL |
| δV1-1.21 | SEQ ID NO: 55 | YDLPSL |
| δV1-1.22 | SEQ ID NO: 56 | YDLGLL |
| δV1-1.23 | SEQ ID NO: 57 | YDLGSI |
| δV1-1.24 | SEQ ID NO: 58 | YDLGSV |
| δV1-1.25 | SEQ ID NO: 59 | IGSL |
| δV1-1.26 | SEQ ID NO: 60 | VGSL |
| δV1-1.27 | SEQ ID NO: 61 | LPSL |
| δV1-1.28 | SEQ ID NO: 62 | LGLL |
| δV1-1.29 | SEQ ID NO: 63 | LGSI |
| δV1-1.30 | SEQ ID NO: 64 | LGSV |
| δV1-2 | SEQ ID NO: 65 | ALSTERGKTLV |
| δV1-2.1 | SEQ ID NO: 66 | ALSTDRGKTLV |
| δV1-2.2 | SEQ ID NO: 67 | ALTSDRGKTLV |
| δV1-2.3 | SEQ ID NO: 68 | ALTTDRGKSLV |
| δV1-2.4 | SEQ ID NO: 69 | ALTTDRPKTLV |
| δV1-2.5 | SEQ ID NO: 70 | ALTTDRGRTLV |
| δV1-2.6 | SEQ ID NO: 71 | ALTTDKGKTLV |
| δV1-2.7 | SEQ ID NO: 72 | ALTTDKGKTL |
| δV1-3 | SEQ ID NO: 73 | VLMRAAEEPV |
| δV1-4 | SEQ ID NO: 74 | QSMRSEDEAK |
| δV1-5 | SEQ ID NO: 75 | AFNSYELGS |
| δV3-1 | SEQ ID NO: 76 | QGFEKKTGV |
| δV3-2 | SEQ ID NO: 77 | DNNGTYGKI |
| δV5-1 | SEQ ID NO: 78 | KNLIDS |
| δV5-2 | SEQ ID NO: 79 | VKSPRDYS |
| δV5-2.1 | SEQ ID NO: 80 | VKSPCRDYS |
| δV5-2.2 | SEQ ID NO: 81 | IKSPRLYS |
| δV5-3 | SEQ ID NO: 82 | KNLIDS |
| δV5-4 | SEQ ID NO: 83 | PKVKSPRDYSN |
| εV1-1 | SEQ ID NO: 84 | NGLLKIK |
| εV1-3 | SEQ ID NO: 85 | LAVFHDAPIGY |
| εV1-4 | SEQ ID NO: 86 | DDFVANCTI |
| εV1-5 | SEQ ID NO: 87 | WIDLEPEGRV |
| εV1-6 | SEQ ID NO: 88 | HAVGPRPQTF |
| εV1-7 | SEQ ID NO: 89 | NGSRHFED |
| εV1-7.1 | SEQ ID NO: 90 | HDAPIGDY |
| εV1-7.2 | SEQ ID NO: 91 | HDAPIG |
| εV1-7.3 | SEQ ID NO: 92 | HDAAIGYD |
| εV1-7.4 | SEQ ID NO: 93 | HDAPIPYD |
| εV1-7.5 | SEQ ID NO: 94 | HNAPIGYD |
| εV1-7.6 | SEQ ID NO: 95 | HAAPIGYD |
| εV1-7.7 | SEQ ID NO: 96 | ADAPIGYD |
| εV1-7.8 | SEQ ID NO: 97 | HDAPAGYD |
| εV1-7.9 | SEQ ID NO: 98 | HDAPIGAD |
| εV1-7.10 | SEQ ID NO: 99 | HDAPIAYD |
| εV1-7.11 | SEQ ID NO: 100 | HDAPIGYA |
| εV3-1 | SEQ ID NO: 101 | SSPSEEDRS |
| εV3-2 | SEQ ID NO: 102 | PCDQEIKE |
| εV3-3 | SEQ ID NO: 103 | ENNIRKALS |
| εV3-4 | SEQ ID NO: 104 | GEVRQGQA |
| εV5-1 | SEQ ID NO: 105 | EAIVKQ |
| εV5-2 | SEQ ID NO: 106 | IKTKRDV |
| εV5-2.1 | SEQ ID NO: 107 | IKTKRLI |
| εV5-3 | SEQ ID NO: 108 | CEAIVKQ |
| εV5-4 | SEQ ID NO: 109 | TKRDVNNFDQ |
| ζV1-1 | SEQ ID NO: 110 | VRLKAHY |
| ζV1-2 | SEQ ID NO: 111 | VDSEGD |
| ζV1-3 | SEQ ID NO: 112 | VFPSIPEQ |
| ζV3-1 | SEQ ID NO: 113 | SQEPPVDDKNEDADL |
| ζV3-2 | SEQ ID NO: 114 | IKDDSED |
| ζV3-3 | SEQ ID NO: 115 | PVIDGMDGI |
| ζV5-1 | SEQ ID NO: 116 | EDAIKR |
| ζV5-1.1 | SEQ ID NO: 117 | EDAIR |
| ζV5-2 | SEQ ID NO: 118 | ITDDYGLD |
| ζV5-2.1 | SEQ ID NO: 119 | ITDDYGDL |
| ζV5-3 | SEQ ID NO: 120 | DDYGLDN |
| ηV1-1 | SEQ ID NO: 121 | NGYLRVR |
| ηV1-2 | SEQ ID NO: 122 | EAVGLQPT |
| ηV1-3 | SEQ ID NO: 123 | LAVFHETPLGY |
| ηV1-4 | SEQ ID NO: 124 | DFVANCTL |
| ηV1-5 | SEQ ID NO: 125 | WVDLEPEGKV |
| ηV1-6 | SEQ ID NO: 126 | HSLFKKGH |
| ηV1-7 | SEQ ID NO: 127 | TGASDTFEG |
| ηV5-1 | SEQ ID NO: 128 | EGHLPM |
| ηV5-1.1 | SEQ ID NO: 129 | EGHDPM |
| ηV5-2 | SEQ ID NO: 130 | IKSREDVS |
| ηV5-3 | SEQ ID NO: 131 | VRSREDVS |
| ηV5-4 | SEQ ID NO: 132 | PRIKSREDV |
| λV1-1 | SEQ ID NO: 133 | HQVRVKAYYR |
| λV1-2 | SEQ ID NO: 134 | YELNKDSELLI |
| λV3-1 | SEQ ID NO: 135 | MDQSSMHSDHAQTVI |
| λV3-2 | SEQ ID NO: 136 | LDQVGEE |
| λV3-3 | SEQ ID NO: 137 | EAMNTRESG |
| λV5-1 | SEQ ID NO: 138 | DDIVRK |
| μV5-2 | SEQ ID NO: 139 | VKLCDFGF |
| μV5-2.1 | SEQ ID NO: 140 | IRLCDFAF |
| μV5-3 | SEQ ID NO: 141 | QVKLCDFGFA |
| μV1-1 | SEQ ID NO: 142 | MSVPPLLRP |
| μV1-2 | SEQ ID NO: 143 | KFPECGFYGLY |
| μV3-1 | SEQ ID NO: 144 | DPDADQEDS |
| μV3-2 | SEQ ID NO: 145 | SKDTLRKRH |
| μV3-3 | SEQ ID NO: 146 | ITLFQNDTG |
| μV3-4 | SEQ ID NO: 147 | GSNSHKDIS |
| μV5-1 | SEQ ID NO: 148 | SDSPEA |
| θV1-1 | SEQ ID NO: 149 | GLSNFDCG |
| θV1-2 | SEQ ID NO: 150 | YVESENGQMYI |
| θV1-3 | SEQ ID NO: 151 | IVKGKNVDLI |
| θV1-4 | SEQ ID NO: 152 | DMNEFETEGF |
| θV3-1 | SEQ ID NO: 153 | CSIKNEARL |
| θV3-2 | SEQ ID NO: 154 | GKREPQGIS |
| θV3-3 | SEQ ID NO: 155 | DEVDKMCHL |
| θV5-1 | SEQ ID NO: 156 | RALINS |
| θV5-2 | SEQ ID NO: 157 | VKSPFDCS |
| θV5-2.1 | SEQ ID NO: 158 | VRSPFDCS |
| θV5-3 | SEQ ID NO: 159 | DRALINS |
| ιV5-1 | SEQ ID NO: 160 | ISGEFGLD |
| ιV5-1.1 | SEQ ID NO: 161 | CSGEFGLD |
| ιV5-2 | SEQ ID NO: 162 | DDDIVRK |
| βI V5-1 | SEQ ID NO: 163 | KLFIMN |
| βII V5-1 | SEQ ID NO: 164 | QEVIRN |
| βI V3-1 | SEQ ID NO: 165 | VPPEGSEA |
| αV5-1 | SEQ ID NO: 166 | QLVIAN |
| HIV TAT Transregulatory Domain | SEQ ID NO: 167 | GRKKRRQRRRPPQC |
| (48-60) | | |
| HIV TAT Transregulatory Domain | SEQ ID NO: 168 | LGISYGRKKRRQRRRPPQC |
| (43-60) | | |
| HIV TAT Transregulatory Domain | SEQ ID NO: 169 | FITKALGISYGRKKRRQRRRPPQC |
| (37-60) | | |
| HIV TAT Transregulatory Domain | SEQ ID NO: 170 | FITKALGISYGRKKRRC |
| (37-53) | | |
| δV1 PKC domain | SEQ ID NO: 171 | MAPFLRISFN SYELGSLQAE |
| | | DDASQPFCAV KMKEALTTDR |
| | | GKTLVQKKPT MYPEWKSTFD |
| | | AHIYEGRVIQ IVLMRAAEDP |
| | | MSEVTVGVSV LAERCKKNNG |
| | | KAEFWLDLQP QAKVLMCVQY |
| | | FLEDGDCKQS MRSEEEAMFP |
| | | TMNRRGAIKQ AKIHYIKNHE |
| δV5 PKC domain | SEQ ID NO: 172 | PKVKSPSDYS NFDPEFLNEK |
| | | PQLSFSDKNL IDSMDQEAFH |
| | | GFSFVNPKFE QFLDI |

### DETAILED DESCRIPTION

The present methods and compositions now will be described more fully hereinafter. This subject matter may, however, be embodied in many different forms and should not be construed as limited to the embodiments set forth herein; rather, these embodiments are provided so that this disclosure will be thorough and complete, and will fully convey the scope of the methods and compositions to those skilled in the art.

All publications, patents and patent applications cited herein, whether supra or infra, are hereby incorporated by reference in their entirety.

### I. Definitions

Unless otherwise indicated, all terms should be given their ordinary meaning as known in the art. See, e.g., Ausubel, F. M. et al., John Wiley and Sons, Inc., Media Pa., for definitions and terms of art. Abbreviations for amino acid residues are the standard 3-letter and/or 1-letter codes used in the art to refer to one of the 20 common L-amino acids.

As used herein and in the appended claims, the singular forms “a”, “an”, and “the” include plural reference unless the context clearly dictates otherwise.

As used herein a “conserved set” of amino acids refers to a contiguous sequence of amino acids that is identical or closely homologous (e.g., having only conservative amino acid substitutions or having a specified percent identity) between two proteins or peptides. A conserved set may be anywhere from 5-50 amino acid residues in length, more preferably from 6-40, still more preferably from 6-20, 8-20, 6-15, or 8-15 residues in length.

As used herein, a “conservative amino acid substitutions” are substitutions that do not result in a significant change in the activity or tertiary structure of a selected polypeptide or protein. Such substitutions typically involve replacing a selected amino acid residue with a different residue having similar physico-chemical properties. For example, substitution of Glu for Asp is considered a conservative substitution since both are similarly-sized negatively-charged amino acids. Groupings of amino acids by physico-chemical properties are known to those of skill in the art and examples are given below.

The terms “peptide” and “polypeptide” are used interchangeably herein and refer to a compound made up of a chain of amino acid residues linked by peptide bonds. Unless otherwise indicated, the sequence for peptides is given in the order from the “N” (or amino) terminus to the “C” (or carboxyl) terminus.

As used herein, the term “intradermal” means that a therapeutically effective amount of PKC modulatory conjugate is applied to skin to deliver the conjugate to layers of skin beneath the stratum corneum.

As used herein, the term “transdermal” means that a therapeutically effective amount of a PKC modulatory conjugate is applied to skin to deliver the conjugate to systemic circulation.

### II. Methods of Administration

In one aspect, methods for administering a compound transdermally are provided. In a preferred embodiment, the compound is attached to a cell-penetrating peptide that facilitates transport of the compound across a cell membrane. Application of the compound-carrier protein conjugate to microporated skin achieves delivery of the conjugate locally, systemically, or both.

In one aspect, methods for administering an PCK modulatory protein or peptide transdermally are provided. In a preferred embodiment, the PKC modulatory protein or peptide is isozyme selective. The methods comprise administering to microporated skin a peptide capable of selective modulation, i.e., activation or inhibition, of a specific PKC isozyme. As will be illustrated below, the isozyme-selective PKC modulatory peptide is administered to microporated skin in the form of a conjugate, where the PKC peptide is attached to a carrier peptide that facilitates transport of the peptide across a cell membrane. As will be further discussed below, and supported by the studies set forth in the Examples, application of the conjugate to microporated skin achieves delivery of the conjugate intradermally and/or transdermally. As noted above, inhibition or activation of a selected PKC isozyme is an intracellular process. Delivery of a peptide or protein to inhibit or activate a PKC isozyme thus requires entry of the peptide or protein into the cell. Transdermal delivery of peptides or proteins for modulation of a PKC isozyme must achieve delivery across the stratum corneum followed by delivery across a cell membrane. As will be shown by the data herein, administration of the PKC modulatory peptide in the form of a conjugate, where the PKC modulatory peptide is linked to a cell-penetrating or carrier peptide, provides delivery of the conjugate intradermally and/or transdermally. It is desirable that the intact conjugate be delivered to the destination (e.g., the blood or a local intradermal site), so that the cell-penetrating peptide portion of the conjugate remains linked to the PKC modulatory peptide to facilitate transport of the PKC modulatory peptide across the cell membrane for activity intracellularly. Heretofore, it was unknown and unpredictable whether such a conjugate delivered transdermally could or would enter and cross through the layers of skin beneath the stratum corneum, and/or be delivered to the systemic circulation.

A. Isozyme Specific PKC Modulatory Peptides

Isozyme specific PKC modulatory peptides are described in the literature, and the sequences of exemplary peptides are provided in the table set forth in the Brief Description of the Sequences above. Several of the specific peptides are mentioned individually merely for illustration of representative PKC modulatory peptides.

In one embodiment, the PKC modulatory peptide has isozyme selective activity for activating or inhibiting epsilon PKC (εPKC). An exemplary εPKC activating peptide is identified as SEQ ID NO: 18 (HDAPIGYD). This epsilon PKC activating peptide promotes translocation of epsilon PKC intracellularly, which is correlated with cytoprotection in human tissues, especially the heart. Administration of SEQ ID NO: 18 prior to or during ischemia is cardioprotective. Other exemplary εPKC peptide inhibitors include, but are not limited to, the following sequences. In a first embodiment, an εPKC peptide inhibitor has a sequence that corresponds to between about 6-20 contiguous amino acid residues from the εPKC first or fifth variable domains, having sequences identified as SEQ ID NO: 1 and SEQ ID NO: 2, respectively. In another embodiment, the εPKC peptide inhibitor has a sequence that has a selected percent identity to between about 6-20 contiguous amino acid residues of SEQ ID NO: 1 or SEQ ID NO: 2. In various embodiments, the selected percent sequence identity is at least about, or equal to, 50%, 55%, 60%, 65%, 70%, 75%, 80%, 85%, 86%, 87%, 88%, 89%, 90%, 91%, 92%, 93%, 94%, 95%, 96%, 97%, 98%, or 99%. In other embodiments, the εPKC peptide inhibitor has a sequence that is at least a selected percent identity with between about 7-18, 8-16, 8-15, 9-15, 10-15 contiguous amino acid residues from SEQ ID NO:1 or SEQ ID NO: 2, including conservative amino acid residue substitutions thereof.

Exemplary εPKC modulatory peptides include, but are not limited to, the sequences identified as SEQ ID NO: 10-28, and to sequences having at least about, or equal to, 50%, 55%, 60%, 65%, 70%, 75%, 80%, 85%, 86%, 87%, 88%, 89%, 90%, 91%, 92%, 93%, 94%, 95%, 96%, 97%, 98%, or 99% percent identity to any of SEQ ID NOs: 10-28. In one embodiment, the εPKC modulatory peptide is an inhibitor of epsilon PKC, and has a sequence that is or has a selected percent sequence identity to EAVSLKPT (εV1-2; SEQ ID NO:13). Inhibition of epsilon-PKC is associated with attenuation of pain, as further discussed below. In another embodiment, the εPKC modulatory peptide is an activator of epsilon PKC, and has a sequence that is or has a selected percent sequence identity to or HDAPIGYD (ψεRACK; SEQ ID NO:18). Activation of epsilon PKC is cardioprotective and reduces damage to tissue caused by ischemia.

In another embodiment, the PKC modulatory peptide has isozyme selective activity for activating or inhibiting gamma PKC (γPKC). Exemplary γPKC peptide inhibitors include, but are not limited to, the following sequences. In a first embodiment, a γPKC peptide inhibitor has a sequence that corresponds to between about 6-20 contiguous amino acid residues from the γPKC first or fifth variable domains, having sequences identified as SEQ ID NO: 3 and SEQ ID NO: 4, respectively. In another embodiment, the γPKC peptide inhibitor has a sequence that has a selected percent identity to between about 6-20 contiguous amino acid residues of SEQ ID NO: 3 or SEQ ID NO: 4. In various embodiments, the selected percent sequence identity is at least about, or equal to, 50%, 55%, 60%, 65%, 70%, 75%, 80%, 86%, 87%, 88%, 89%, 90%, 91%, 92%, 93%, 94%, 95%, 96%, 97%, 98%, or 99%. In other embodiments, the γPKC peptide inhibitor has a sequence that is at least a selected percent identity with between about 7-18, 8-16, 8-15, 9-15, or 10-15 contiguous amino acid residues from SEQ ID NO: 3 or SEQ ID NO: 4, including conservative amino acid residue substitutions thereof.

Exemplary γPKC peptide inhibitors include, but are not limited to the sequences identified as SEQ ID NO: 29-45, and to sequences having at least about, or equal to, 50%, 55%, 60%, 65%, 70%, 75%, 80%, 85%, 86%, 87%, 88%, 89%, 90%, 91%, 92%, 93%, 94%, 95%, 96%, 97%, 98%, or 99% percent identity to any of SEQ ID NOs: 29-45. In one embodiment, the γPKC modulatory peptide is an inhibitor, and in preferred embodiments, the selective γPKC inhibitor peptide has a sequence that is or has a selected percent sequence identity to RLVLAS (SEQ ID NO:43). Inhibition of gamma PKC is associated with attenuation of pain.

Other peptides for selective modulation, inhibition or activation, of εPKC and γPKC isozymes are identified herein, and are known in the art (see, e.g., U.S. Pat. Nos. 5,783,405; 6,686,334; 6,165,977, 6,855,693; and 7,459,424; and U.S. Publication Nos. 2004/0204364; 2002/0150984; 2002/0168354; 2002/057413; 2003/0223981; and 2004/0009922

In another embodiment, the PKC modulatory peptide has isozyme selective activity for activating or inhibiting delta PKC (δPKC). Exemplary peptides with activity to selectively modulate δPKC are identified as SEQ ID NOs: 50 and 52-83. Peptides having at least about, or equal to, 50%, 55%, 60%, 65%, 70%, 75%, 80%, 85%, 86%, 87%, 88%, 89%, 90%, 91%, 92%, 93%, 94%, 95%, 96%, 97%, 98%, or 99% percent identity to any of SEQ ID NOs: 50 and 52-83. In one embodiment, a peptide with activity to selectively modulate δPKC has at least about, or equal to, 50%, 55%, 60%, 65%, 70%, 75%, 80%, 85%, 86%, 87%, 88%, 89%, 90%, 91%, 92%, 93%, 94%, 95%, 96%, 97%, 98%, or 99% percent identity to 6-20, 8-20, 6-15, or 8-15 contiguous residues of SEQ ID NO: 171 or SEQ ID NO: 172. In another embodiment, the δPKC modulatory peptide is an inhibitor of δPKC, and in preferred embodiments, the selective δPKC inhibitor peptide has a sequence that is or has a selected percent sequence identity to SFNSYELGSL (δV1-1; SEQ ID NO:50). Inhibition of delta PKC is associated with attenuation of ischemic injury to tissue and with reduction of tissue injury during reperfusion. In another embodiment, the δPKC modulatory peptide is an activator of δPKC. Activation of δPKC is associated with apoptosis and can potentiate the effect of chemotherapeutics, both desirable for cancer therapy.

In another embodiment, the PKC modulatory peptide has isozyme selective activity for activating or inhibiting beta PKC (βPKC), and more specifically βIPKC and/or βIIPKC. Peptides with activity to selectively modulate βPKC are identified as SEQ ID NOs: 163, 164 and 165. Peptides having at least about, or equal to, 50%, 55%, 60%, 65%, 70%, 75%, 80%, 85%, 86%, 87%, 88%, 89%, 90%, 91%, 92%, 93%, 94%, 95%, 96%, 97%, 98%, or 99% percent identity to 6-20, 8-20, 6-15, or 8-15 contiguous residues of the full βIPKC and/or βIIPKC PKC sequences, or with any of SEQ ID NOs: 163-165, are contemplated. In one embodiment, the βPKC modulatory peptide is an inhibitor of βPKC, and in another embodiment, the βPKC modulatory peptide is an activator of βPKC. Inhibition of βPKC is associated with anti-angiogenesis, which has therapeutic applications in oncology, age-related macular degeneration and diabetic retinopathy.

In another embodiment, the PKC modulatory peptide has isozyme selective activity for activating or inhibiting alpha PKC (αPKC). An exemplary peptide with activity to selectively modulate αPKC is identified as SEQ ID NO: 166. Peptides having at least about, or equal to, 50%, 55%, 60%, 65%, 70%, 75%, 80%, 85%, 86%, 87%, 88%, 89%, 90%, 91%, 92%, 93%, 94%, 95%, 96%, 97%, 98%, or 99% percent identity to 6-20, 8-20, 6-15, or 8-15 contiguous residues of the full sequence of αPKC, or with SEQ ID NO: 166, are contemplated. In one embodiment, the αPKC modulatory peptide is an inhibitor of αPKC, and in another embodiment, the αPKC modulatory peptide is an activator of αPKC. Inhibition of αPKC is associated with inhibition of metastasis, which has therapeutic application in oncology.

In another embodiment, the PKC modulatory peptide has isozyme selective activity for activating or inhibiting theta PKC (θPKC), and more specifically with inhibition of θPKC. Exemplary peptides with activity to selectively modulate θPKC are identified as SEQ ID NOs: 150-160. Peptides having at least about, or equal to, 50%, 55%, 60%, 65%, 70%, 75%, 80%, 85%, 86%, 87%, 88%, 89%, 90%, 91%, 92%, 93%, 94%, 95%, 96%, 97%, 98%, or 99% percent identity to 6-20, 8-20, 6-15, or 8-15 contiguous residues of the full sequence of θPKC, or with any one of SEQ ID NOs: 150-160, are contemplated. In one embodiment, the θPKC modulatory peptide is an inhibitor of θPKC, and in another embodiment, the θPKC modulatory peptide is an activator of θPKC. Inhibition of θPKC is associated with immune modulation, which has therapeutic application in immune suppression.

Generally, peptides with inhibitory activity for a selected isozyme of PKC may be identified using assays that measure the activation, intracellular translocation, binding to intracellular receptors (e.g. RACKs) or catalytic activity of the respective PKC. Traditionally, the kinase activity of PKC family members has been assayed using at least partially purified PKC in a reconstituted phospholipid environment with radioactive ATP as the phosphate donor and a histone protein or a short peptide as the substrate (Kitano, M. et al, Meth. Enzymol., 124:349-352 (1986); Messing, R. O. et al, J. Biol. Chem., 266:23428-23432 (1991)). Exemplary assays are a rapid, highly sensitive chemiluminescent assay that measures protein kinase activity at physiological concentrations and can be automated and/or used in high-throughput screening (Lehel, C. et al, Anal. Biochem., 244:340-346 (1997)) and an assay using PKC in isolated membranes and a selective peptide substrate that is derived from the MARCKS protein (Chakravarthy, B. R. et al, Anal. Biochem., 196:144-150 (1991)). Inhibitors that affect the intracellular translocation of a PKC can be identified by assays in which the intracellular localization of the PKC is determined by fractionation (Messing, R. O. et al., J. Biol. Chem., 266:23428-23432 (1991)) or immunohistochemistry (U.S. Pat. No. 5,783,405; U.S. Pat. No. 6,255,057). The selectivity of such PKC inhibitors can be determined by comparing the effect of the inhibitor on the particular PKC with its effect on other PKC isozymes.

It will be appreciated that conservative amino acid substitutions may be made in the amino acid sequences to obtain modified peptides of those described herein. Conservative amino acid substitutions, as known in the art and as referred to herein, involve substituting amino acids in a protein or peptide with amino acids having similar side chains in terms of, for example, structure, size and/or chemical properties. For example, the amino acids within each of the following groups may be interchanged with other amino acids in the same group: amino acids having aliphatic side chains, including glycine, alanine, valine, leucine and isoleucine; amino acids having non-aromatic, hydroxyl-containing side chains, such as serine and threonine; amino acids having acidic side chains, such as aspartic acid and glutamic acid; amino acids having amide side chains, including glutamine and asparagine; basic amino acids, including lysine, arginine and histidine; amino acids having aromatic ring side chains, including phenylalanine, tyrosine and tryptophan; and amino acids having sulfur-containing side chains, including cysteine and methionine. Additionally, aspartic acid, glutamic acid and their amides, are also considered interchangeable herein.

Percent identity may be determined, for example, by comparing sequence information using the advanced BLAST computer program, including version 2.2.9, available from the National Institutes of Health. The BLAST program is based on the alignment method of Karlin and Altschul. Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the proteins being compared. Default parameters are provided to optimize searches with short query sequences in, for example, blastp with the program. The program also allows use of an SEG filter to mask-off segments of the query sequences as determined by the SEG program of Wootton and Federhen, Computers and Chemistry 17:149-163 (1993).

The modulatory peptide may include natural amino acids, such as the L-amino acids or non-natural amino acids, such as D-amino acids. The amino acids in the peptide may be linked by peptide bonds or, in modified peptides described herein, by non-peptide bonds.

A skilled artisan will appreciate that isozyme selective modulatory peptides can be identified from the PKC sequences of various species. For example, the peptides of the first and fifth variable domains of εPKC and γPKC identified above as SEQ ID NOs: 1-4 are human sequences, it is understood that the first and fifth variable domains of εPKC and γPKC from other species are contemplated, and several examples of sequences from Rattus norvegicus are identified as SEQ ID NOs: 46-48.

B. Cell Penetrating Peptide and Conjugate

As noted above, the PKC modulatory peptide is administered transdermally in the form of a conjugate, where the PKC modulatory peptide is modified by attachment to another peptide or to a linker to form a fusion peptide or a conjugate. For example, the peptide modulators can be modified by one or more C-terminal or N-terminal amino acid residues, such as a Cys, to form a reactive site for cross-linking to another peptide. As a further example, the peptide modulator may be linked or otherwise conjugated to a second peptide by an amide bond from the C-terminal of one peptide to the N-terminal of the other peptide. The linkage between the inhibitor peptide and the other peptide may be a non-cleavable peptide bond, or a cleavable bond, such as an ester or other cleavable bond known to the art. The peptide attached to the PKC modulatory peptide can be a peptide that functions to increase the cellular uptake of the peptide inhibitors, has another desired biological effect, such as a therapeutic effect, or may have both of these functions. For example, it may be desirable to conjugate, or otherwise attach, a PKC modulatory inhibitory peptide to a cytokine or other protein that elicits a desired biological response.

In one embodiment, the modulatory peptide is attached to a carrier peptide, such as a cell permeable carrier peptide. The cell permeable carrier peptide functions to facilitate cellular uptake of the PKC modulatory peptide, and may be, for example, a Drosophila Antennapedia homeodomain-derived sequence which is set forth in SEQ ID NO:5 (CRQIKIWFQNRRMKWKK), which may be attached to the PKC modulatory peptide by cross-linking via an N-terminal Cys-Cys bond (Theodore, L., et al. J. Neurosci. 15:7158-7167 (1995); Johnson, J. A., et al. Circ. Res 79:1086 (1996)).

Alternatively, the PKC modulatory peptide may be modified by attachment to a Transactivating Regulatory Protein (Tat)-derived transport polypeptide (such as from amino acids 47-57 of Tat shown in SEQ ID NO:6 (YGRKKRRQRRR) from the Human Immunodeficiency Virus, Type 1, (Vives et al., J. Biol. Chem., 272:16010-16017 (1997), U.S. Pat. No. 5,804,604 and Genbank Accession No. AAT48070). Various other sequences and fragments of Tat are described in the art (Vives et al., supra) and are contemplated for use herein as the cell-penetrating peptide portion of the conjugate. For example, SEQ ID NO: 167-170 correspond to fragments of Tat that promote intracellular delivery of covalently bound peptides. A skilled artisan will appreciate in view of the disclosure herein and in view of the art that other cell-penetrating peptides can be identified, and would be suitable for use in the methods described herein. For example, a peptide having 6-20 contiguous amino acid residues, more preferably 8-15 contiguous amino acid residues, 6-11 contiguous amino acid residues or 8-11 contiguous amino acid residues, from SEQ ID NO: 6 would be suitable for a carrier peptide. In another embodiment, a carrier peptide having at least about, or equal to, 50%, 55%, 60%, 65%, 70%, 75%, 80%, 85%, 86%, 87%, 88%, 89%, 90%, 91%, 92%, 93%, 94%, 95%, 96%, 97%, 98%, or 99% percent identity to 6-20, 8-15, 6-15, 6-15 or 6-11 contiguous residues of SEQ ID NO:6 or SEQ ID NO: 5 are contemplated for use as a carrier peptide.

In another embodiment, the PKC modulatory peptide is modified by attachment to a polyarginine peptide (Mitchell et al., J. Peptide Res. 56:318-325 (2000); Rothbard et al., Nature Med. 6:1253-1257 (2000); U.S. Pat. No. 6,593,292), where in some embodiments the polyarginine peptide has between 6-25 sequential arginine residues. In one embodiment, the polyarginine is not octa-arginine, and in another embodiment the polyarginine carrier peptide is not hepta-arginine, and in another embodiment the polyarginine carrier peptide is not hexa-arginine. The inhibitors may be modified by other methods known to the skilled artisan in order to increase the cellular uptake of the inhibitors.

The conjugate and its method of transdermal delivery may be discussed in terms of a peptide for modulation of a specific PKC isozyme, a skilled artisan will readily recognized, and indeed the inventors have contemplated, conjugates prepared from a peptide having modulatory activity for any of the PKC isozymes. Examples and discussions herein that are particular to a specific PKC modulatory peptide are merely exemplary of the peptides for any given PKC isozyme. With this understanding, and based on the description above of exemplary cell penetrating peptides and approaches for linking to a PKC modulatory peptide, a skilled artisan can readily envision conjugates for use in the methods described herein. For purposes of illustration, exemplary peptide-carrier protein conjugates are identified herein as SEQ ID NO: 14, SEQ ID NO: 19, SEQ ID NO: 49 and SEQ ID NO:51. The conjugate identified as SEQ ID NO: 14 is comprised of the eight amino acid residue inhibitor peptide εV1-2 (SEQ ID NO: 13) and an arginine-rich eleven amino acid carrier peptide (SEQ ID NO: 6) derived from the TAT protein. The two peptides are attached by a two amino acid residue linker, GG. It will be appreciated that the amino acid linker can be any number of residues (2, 3, 4, 5, 6, 7, 8 residues, or between for example 2-8, 3-7, or 3-6 residues), where glycine is merely exemplary. The conjugate identified as SEQ ID NO: 19 is comprised of the isozyme specific peptide εV1-7 (SEQ ID NO: 18) and a carrier peptide (SEQ ID NO: 6) attached by a 6-amino-1-hexanoic acid chemical linking moiety. It will be appreciated that other chemical linking moieties are contemplated, and a skilled artisan can readily identify other suitable moieties. The εV1-7 peptide has an acyl (Ac) end cap, in this embodiment at the N-terminus of the peptide, to enhance its stability, and the C-terminus of the conjugate is amidated (NH₂). Additional examples of linking moieties and chemical moieties for placement at the C-terminus or N-terminus of the conjugates are described in U.S. Publication No. 2009/0042769. The conjugate identified as SEQ ID NO: 51 is comprised of the ten amino acid residue modulatory peptide δV1-1 (SEQ ID NO: 50) and an arginine-rich eleven amino acid carrier peptide (SEQ ID NO: 6) derived from the TAT protein. The two peptides are attached by a disulphide bond between Cys residues added to the N′ terminus of each peptide.

Modifications to the PKC modulatory peptide or to the carrier peptide, or to both, to increase the stability and/or delivery efficiency of the conjugate, by for example, reducing disulfide bond exchange, physical stability, reducing proteolytic degradation, and/or increasing efficiency of cellular uptake, are contemplated. The stability of the disclosed inhibitory peptides and conjugates may be improved through the use of chemical modifications and/or by controlling the physical environment of the peptide compositions prior to use. Such chemical modifications are well-known and are described in U.S. Publication No. 2009/0042769.

For example, the joining sulfur-containing residue can be placed anywhere in the sequence of the carrier or cargo peptides. For example, an inhibitory peptide composition may typically have the joining sulfur-containing residue at the amino terminus of the carrier and cargo peptides. The joining sulfur-containing residues may be placed at the carboxy termini of the peptides, or alternatively at the amino terminus of peptide and at the carboxy terminus of the other peptide. Additionally, the joining sulfur-containing residue may be placed anywhere within the sequence of either or both of the peptides. Placing the joining sulfur-containing residue within the carrier peptide, the cargo peptide, or both has been observed to reduce the rate of disulfide bond exchange.

An example of chemical modifications useful to stabilize the disulfide bonds of the inhibitory peptide compositions involves optimizing the amino acid residue or residues immediately proximate to the sulfur-containing residues used to join the carrier and cargo peptide. One method of stabilizing the disulfide bond involves placing an aliphatic residue immediately proximate to the sulfur-containing residue in the carrier and/or cargo peptides. Aliphatic residues include alanine, valine, leucine and isoleucine. Thus, when the joining sulfur-containing residue is placed at the amino terminus of a peptide, an aliphatic residue is placed at the penultimate amino terminal position of the peptide to reduce the rate of disulfide bond exchange. When the joining sulfur-containing residue is located at the carboxy terminus of a peptide, an aliphatic residue is placed at the penultimate carboxy terminal position of the peptide to reduce the rate of disulfide bond exchange. When the joining sulfur-containing residue is located within the sequence of a peptide, the aliphatic residue can be place at either the amino terminal or carboxy terminal side of the residue, or at both sides.

A variety of sulfur-containing residues are contemplated for use. Cysteine and cysteine analogs can also be used as the joining cysteine residues in the peptide composition. Particular cysteine analogs include D-cysteine, homocysteine, alpha-methyl cysteine, mercaptopropionic acid, mercaptoacetic acid, penicillamine, acetylated forms of those analogs capable of accepting an acetyl group, and cysteine analogs modified with other blocking groups. For example, the use of homocysteine, acetylated homocysteine, penicillamine, and acetylated penicillamine in the cargo, the carrier, or both peptides have been shown to stabilize the peptide composition and decrease disulfide bond exchange. Alpha-methyl cysteine inhibits disulfide degration because the base-mediated abstraction of the alpha hydrogen from one cysteine is prevented by the presence of the sulfur atom. Cargo/carrier peptide conjugates joined by disulfide bonds have been shown to be more resistant to glutathione reduction than unmodified peptides. Other cysteine analogs are also useful as joining cysteines. Similarly, stereoisomers of cysteine will inhibit disulfide bond exchange.

Disulfide bond exchange can be eliminated completely by linking the carrier and cargo peptides to form a single, linear peptide. This method is discussed in U.S. Patent Application Publication No. 2009/0042769.

The physical environment of the peptide may also have an effect on stability. For example, stability increases in solution as the pH of the solution decreases (acidic environment better than basic), the temperature of the solution decreases, and as the concentration of the peptide composition in solution decreases. In the lyophilized form, stability increases as the pH decreases, the temperature decreases, and the ratio of the peptide composition to excipient increases. Exemplary excipients are discussed in U.S. Pat. No. 7,265,092.

A number of factors impact the efficiency with which a PKC modulatory peptide or a conjugate thereof is taken up by a target cell. For example, the solubility of the PKC modulatory peptide impacts the efficiency with which the peptide is taken up by a target cell. In turn, the amino acid sequence of a carrier or “cargo” (PKC modulatory peptide) peptide largely determines that solubility the peptide compositions in which they are used. Some peptides, particularly cargo peptides, will contain hydrophobic residues, (e.g., Phe, Tyr, Leu), with regular spacing which allows for intramolecular interactions by a “zipper” mechanism leading to aggregation. The solubility of such peptides can be improved by making certain modifications to the inhibitory peptide sequence. For example, the introduction of solubilizing groups at amino and or carboxy termini or on internal residues, such as hydrating groups, like polyethylene glycol (PEG), highly charged groups, like quaternary ammonium salts, or bulky, branched chains of particular amino acid residues will improve the solubility of peptides. Additionally, those hydrophobic side chains that are shown not to be required for activity can be eliminated by deletion or substitution with a conservative or non-interfering residue, such as an alanine, glycine, or serine, thus improving the solubility of the peptides.

Blood and plasma contain proteases may degrade the PKC modulatory peptides or the carrier peptides which facilitate the cellular uptake of the peptide, or both. One method to decrease proteolytic degradation of the carrier or cargo peptides is to mask the targets of the proteases presented by the peptide composition. Once the PKC modulatory peptide enters the plasma of a subject, it may become vulnerable to attack by peptidases. Strategies that address peptide degradation caused by exopeptidases (any of a group of enzymes that hydrolyze peptide bonds formed by the terminal amino acids of peptide chains) or endopeptidases (any of a group of enzymes that hydrolyze peptide bonds within the long chains of protein molecules) are contemplated, and noted below. Exopeptidases are enzymes that cleave amino acid residues from the amino or carboxy termini of a peptide or protein, and can cleave at specific or non-specific sites. Endopeptidases, which cleave within an amino acid sequence, can also be non-specific, however endopeptidases frequently recognize particular amino sequences (recognition sites) and cleaves the peptide at or near those sites.

One approach for protecting peptide compositions from proteolytic degradation involves the “capping” the amino and/or carboxy termini of the peptides. The term “capping” refers to the introduction of a blocking group to the terminus of the peptide via a covalent modification. Suitable blocking groups serve to cap the termini of the peptides without decreasing the biological activity of the peptides. Acetylation of the amino termini of the described peptides is one method of protecting the peptides from proteolytic degradation. Other capping moieties are possible. The selection of acylating moiety provides an opportunity to “cap” the peptide as well as adjust the hydrophobicity of the compound. For example, the hydrophobicity increases for the following acyl group series: formyl, acetyl, propanoyl, hexanoyl, myristoyl, and are also contemplated as capping moieties. Amidation of the carboxy termini of the described peptides is also a method of protecting the peptides from proteolytic degradation.

Protecting peptides from endopeptidases typically involves identification and elimination of an endopeptidase recognition site from a peptide. Protease recognition cites are well known to those of ordinary skill in the art. Thus it is possible to identify a potential endoprotease recognition site and then eliminating that site by altering the amino acid sequence within the recognition site. Residues in the recognition sequence can be moved or removed to destroy the recognition site. In one embodiment, a conservative substitution is made with one or more of the amino acids which comprise an identified protease recognition site. The side chains of these amino acids possess a variety of chemical properties.

In addition to the modifications discussed above, improved utility for the disclosed modulatory peptide conjugates may be achieved by altering the linkage of the carrier and cargo peptides. For example, in one embodiment, carrier and cargo peptides may be linked to form a linear peptide; for example the species may be linked by a peptide bond to form a linear peptide. Stability and potency of the PKC modulatory peptides can also be increased through the construction of peptide multimers, wherein a plurality of cargo peptides is linked to one or more carrier peptides. An additional embodiment involving a cleavable linker sequence is also contemplated.

Another strategy to improve conjugate stability involves joining the PKC modulatory cargo peptide and the carrier peptide into a single fusion peptide, as opposed to joining the peptides via a disulfide cross-linking bond. For example, the C-terminus of cargo may be linked to the N-terminus of the carrier via the linker. However, the other possible permutations are also contemplated, including linking the peptide via their C-termini, their N-termini, and where the carrier peptide is located at the N-terminal portion of the peptide composition.

Additionally, the steps discussed above to stabilize a disulfide bond linked conjugate can also be used with a linear peptide conjugate, where appropriate. For example, a linear peptide conjugate may be capped at both its amino and carboxy termini. Moreover sequences within the peptide may be scrambled or substituted with D-amino acids.

Another method of improving stability and potency is available by forming multimers with a plurality of cargo peptides associated with one or more carrier peptides. Branched, multivalent peptide compositions will increase avidity, potency and stability of the compositions. By engineering cleavage sites or other release mechanisms into the multimer compositions, the multiple conjugates can release nearly simultaneously, PKC modulatory cargo peptides inside a target cell. An example of multimeric peptides is discussed in Yu et al., J. Biol. Chem., 275(6):3943-9 (2000).

C. Transdermal Administration

The PKC modulatory peptides are administered across the stratum corneum, and/or other layers of the epidermis, for local or systemic delivery. In one embodiment, the PKC modulatory peptide, which may be modified by any one or more of the approaches noted above, is delivered via microporation. Any one of a number of techniques for microporation is contemplated, and several are briefly described.

Microporation can be achieved by mechanical means and/or external driving forces, to breach the stratum corneum to deliver the peptide conjugates described herein through the surface of the skin and into the underlying skin layers and/or the bloodstream. In a first embodiment, the microporation technique is ablation of the stratum corneum in a specific region of the skin using a pulsed laser light of wavelength, pulse length, pulse energy, pulse number, and pulse repetition rate sufficient to ablate the stratum corneum without significantly damaging the underlying epidermis. The PKC modulatory peptide is then applied to the region of ablation. Another laser ablation microporation technique, referred to as laser-induced stress waves (LISW), involves broadband, unipolar and compressible waves generated by high-power pulsed lasers. The LISWs interact with tissues to disrupt the lipids in the stratum corneum, creating intercellular channels transiently within the stratum corneum. These channel, or micropores, in the stratum corneum permit entry of the PKC modulatory peptides.

Sonophoresis or phonophoresis is another microporation technique that uses ultrasound energy. Ultrasound is a sound wave possessing frequencies above 20 KHz. Ultrasound can be applied either continuously or pulsed, and applied at various frequency and intensity ranges (Nanda et al., Current Drug Delivery, 3:233 (2006)).

Another microporation technique involves the use of a microneedle array. The array of microneedles when applied to a skin region on a subject pierce the stratum corneum and do not penetrate to a depth that significantly stimulates nerves or punctures capillaries. The patient, thus, feels no or minimal discomfort or pain upon application of the microneedle array for generation of micropores through which the PKC modulatory peptide in the form of a conjugate is delivered.

Microneedle arrays comprised of hollow or solid microneedles are contemplated, where the PKC modulatory conjugate can be coated on the external surface of the needles, dispensed from the interior of hollow needles or included in the matrix from which the needles are fabricated. Examples of microneedle arrays are described, for example, in Nanda et al., Current Drug Delivery, 3:233 (2006) and Meidan et al. American J. Therapeutics, 11:312 (2004). First generation microneedle arrays were comprised of solid, silicon microneedles that were externally coated with a therapeutic agent. When the microarray of needles was pressed against the skin and removed after about 10 seconds, the permeation of the agent on the needles into the body was readily achieved. Second generation microneedle arrays were comprised of microneedles of hollow silicon or titanium and filled with a solution of the therapeutic conjugate. Newer generations of microneedle arrays are prepared from dissolvable or biodegradable polymers, where the tips of the needles containing the therapeutic conjugate remain in the stratum corneum and slowly dissolve.

The microneedles can be constructed from a variety of materials, including metals, ceramics, semiconductors, organics, polymers, and composites. Exemplary materials of construction include pharmaceutical grade stainless steel, gold, titanium, nickel, iron, tin, chromium, copper, palladium, platinum, alloys of these or other metals, silicon, silicon dioxide, and polymers. Representative biodegradable polymers include polymers of hydroxy acids such as lactic acid and glycolic acid polylactide, polyglycolide, polylactide-co-glycolide, and copolymers with PEG, polyanhydrides, poly(ortho)esters, polyurethanes, poly(butyric acid), poly(valeric acid), and poly(lactide-co-caprolactone). Representative non-biodegradable polymers include polycarbonate, polyester, and polyacrylamides.

The microneedles can have straight or tapered shafts. In one embodiment, the diameter of the microneedle is greatest at the base end of the microneedle and tapers to a point at the end distal the base. The microneedle can also be fabricated to have a shaft that includes both a straight (untapered) portion and a tapered portion. The needles may also not have a tapered end at all, i.e. they may simply be cylinders with blunt or flat tips. A hollow microneedle that has a substantially uniform diameter, but which does not taper to a point, is referred to herein as a “microtube.” As used herein, the term “microneedle” includes both microtubes and tapered needles unless otherwise indicated.

Electroporation is another technique for creating micropores in the skin. This approach uses the application of microsecond or millisecond long high-voltage electrical pulses to created transient, permeable pores within the stratum corneum. Other microporation techniques include use of radio waves to create microchannels in the skin. Thermal ablation is yet another approach to achieve transdermal delivery of the conjugates described herein.

In one embodiment, the skin of a subject is microporated by any one of the techniques above and the therapeutic PKC modulatory conjugate is applied to the microporated skin. The PKC modulatory conjugate can be applied to the microporated skin and is retained in contact with the microporated skin for a desired period of time—e.g., at least about 1 hour, 2 hours, 4 hours, 6 hours, 12 hours, 18 hours, 24 hours, 2 days, 1 week, etc. The conjugate can be contained in a traditional transdermal drug delivery device, in one embodiment. Transdermal devices are known in the art, and typically comprise a backing member that defines a drug reservoir and a means to secure the drug reservoir to the skin. In some cases, the drug reservoir and the means to secure are the same, in that an adhesive layer is loaded with the therapeutic conjugate. In other embodiments, a liquid or gel contains the therapeutic conjugate that is secured to the skin with an adhesive.

In one embodiment a system for transdermal administration of the conjugate comprises: (a) at least one drug reservoir containing the conjugate and, optionally, a pharmaceutically acceptable inorganic or organic base in an amount effective to enhance the flux of the conjugate through the body surface without causing damage thereto; (b) a means for maintaining the system in conjugate transmitting relationship to the body surface and forming a body surface-system interface; and (c) a backing layer that serves as the outer surface of the device during use. In one embodiment, the drug reservoir comprises a polymeric matrix of a pharmaceutically acceptable adhesive material that serves to affix the system to the skin during drug delivery; typically, the adhesive material is a pressure-sensitive adhesive (PSA) that is suitable for long-term skin contact, and which should be physically and chemically compatible with the active agent, inorganic or organic base, and any carriers, vehicles or other additives that are present. Examples of suitable adhesive materials include, but are not limited to, the following polyethylenes; polysiloxanes; polyisobutylenes; polyacrylates; polyacrylamides; polyurethanes; plasticized ethylene-vinyl acetate copolymers; and tacky rubbers such as polyisobutene, polybutadiene, polystyrene-isoprene copolymers, polystyrene-butadiene copolymers, and neoprene (polychloroprene). Preferred adhesives are polyisobutylenes.

The backing layer functions as the primary structural element of the transdermal system and provides the device with flexibility and, preferably, occlusivity. The material used for the backing layer should be inert and incapable of absorbing the drug, the base enhancer, or other components of the formulation contained within the device. The backing is preferably comprised of a flexible elastomeric material that serves as a protective covering to prevent loss of drug and/or vehicle via transmission through the upper surface of the patch, and will preferably impart a degree of occlusivity to the system, such that the area of the body surface covered by the patch becomes hydrated during use. The material used for the backing layer should permit the device to follow the contours of the skin and be worn comfortably on areas of skin such as at joints or other points of flexure, that are normally subjected to mechanical strain with little or no likelihood of the device disengaging from the skin due to differences in the flexibility or resiliency of the skin and the device. The materials used as the backing layer are either occlusive or permeable, as noted above, although occlusive backings are preferred, and are generally derived from synthetic polymers (e.g., polyester, polyethylene, polypropylene, polyurethane, polyvinylidine chloride, and polyether amide), natural polymers (e.g., cellulosic materials), or macroporous woven and nonwoven materials.

During storage and prior to use, the laminated structure preferably includes a release liner. Immediately prior to use, this layer is removed from the device so that the system may be affixed to the microporated skin. The release liner should be made from a conjugate/vehicle impermeable material, and is a disposable element, which serves only to protect the device prior to application. Typically, the release liner is formed from a material impermeable to the pharmacologically active agent and the base enhancer, and is easily stripped from the transdermal patch prior to use.

Additional layers, e.g., intermediate fabric layers and/or rate-controlling membranes, may also be present in any of these drug delivery systems. Fabric layers may be used to facilitate fabrication of the device, while a rate-controlling membrane may be used to control the rate at which a component permeates out of the device. The component may be a drug, a base enhancer, an additional enhancer, or some other component contained in the drug delivery system.

Generally, the underlying surface of the transdermal device, i.e., the skin contact area, has an area in the range of about 5-200 cm², preferably 5-100 cm², more preferably 20-60 cm². That area will vary, of course, with the amount of conjugate to be delivered and the flux of the conjugate through the microporated skin.

Such drug delivery systems may be fabricated using conventional coating and laminating techniques known in the art. For example, adhesive matrix systems can be prepared by casting a fluid admixture of adhesive, drug and vehicle onto the backing layer, followed by lamination of the release liner. Similarly, the adhesive mixture may be cast onto the release liner, followed by lamination of the backing layer. Alternatively, the drug reservoir may be prepared in the absence of drug or excipient, and then loaded by soaking in a conjugate/vehicle mixture. In general, transdermal systems of the invention are fabricated by solvent evaporation, film casting, melt extrusion, thin film lamination, die cutting, or the like. The inorganic or organic base permeation enhancer will generally be incorporated into the device during patch manufacture rather than subsequent to preparation of the device. Thus, for acid addition salts of basic drugs (e.g., hydrochloride salts of amine drugs), the enhancer will neutralize the drug during manufacture of the drug delivery system, resulting in a final drug delivery system in which the drug is present in nonionized, neutral form along with an excess of base to serve as a permeation enhancer. For nonionized acidic drugs, the base will neutralize such drugs by converting them to the ionized drug in salt form.

Other types and configurations of transdermal drug delivery systems may also be used in conjunction with the method of the present invention, as will be appreciated by those skilled in the art of transdermal drug delivery. See, for example, Ghosh, Transdermal and Topical Drug Delivery Systems (Interpharm Press, 1997), particularly Chapters 2 and 8.

In another embodiment, the conjugate is applied to the microporated skin in the form of a cream, lotion, ointment, gel, paste, and the like. Ointments, as is well known in the art of pharmaceutical formulation, are semisolid preparations that are typically based on petrolatum or other petroleum derivatives. The specific ointment foundation to be used, as will be appreciated by those skilled in the art, is one that will provide for optimum drug delivery, and, preferably, will provide for other desired characteristics as well, e.g., emolliency or the like. As with other carriers or vehicles, the ointment foundation should be inert, stable, nonirritating and nonsensitizing. As explained in Remington: The Science and Practice of Pharmacy, 20.sup.th edition (Lippincott Williams & Wilkins, 2000), ointment foundations may be grouped in four classes: oleaginous, emulsifiable, emulsion, and water-soluble. Oleaginous ointment foundations include, for example, vegetable oils, fats obtained from animals, and semisolid hydrocarbons obtained from petroleum. Emulsifiable ointment foundations, also known as absorbent ointment foundations, contain little or no water and include, for example, hydroxystearin sulfate, anhydrous lanolin and hydrophilic petrolatum. Emulsion ointment foundations are either water-in-oil (W/O) emulsions or oil-in-water (O/W) emulsions, and include, for example, cetyl alcohol, glyceryl monostearate, lanolin and stearic acid. Preferred water-soluble ointment foundations are prepared from polyethylene glycols of varying molecular weight.

Creams, as also well known in the art, are viscous liquids or semisolid emulsions, either oil-in-water or water-in-oil. Cream foundations are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

As will be appreciated by those working in the field of pharmaceutical formulation, gels are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the “carbomer” family of polymers, e.g., carboxypolyalkylenes that may be obtained commercially under the CARBOPOL®. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added.

Lotions are preparations to be applied to the skin surface and are typically liquid or semiliquid preparations in which solid particles, including the active conjugate, are present in a water or alcohol base. Lotions are usually suspensions of solids, and preferably, for the present purpose, comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

Pastes are semisolid dosage forms in which the active agent is suspended in a suitable foundation. Depending on the nature of the foundation, pastes are divided between fatty pastes or those made from single-phase, aqueous gels. The foundation in a fatty paste is generally petrolatum or hydrophilic petrolatum or the like. The pastes made from single-phase aqueous gels generally incorporate carboxymethylcellulose or the like as the foundation.

In another embodiment, the conjugate is transdermally transported effectively using iontophoresis. In iontophoresis, ions bearing a positive charge are driven across the skin at the site of an electrolytic electrical system anode while ions bearing a negative charge are driven across the skin at the site of an electrolytic system cathode. Integrated devices referred to in the art as “iontophoretic transdermal patch” allow for the administration of a therapeutic compound, such as the modulatory conjugates described herein, through the skin by using electrical current to promote the absorption of the conjugate from the patch through the skin of the subject. The patch typically comprises electrical components, the therapeutic conjugate, and an adhesive backing layer.

D. Examples of Transdermal Delivery

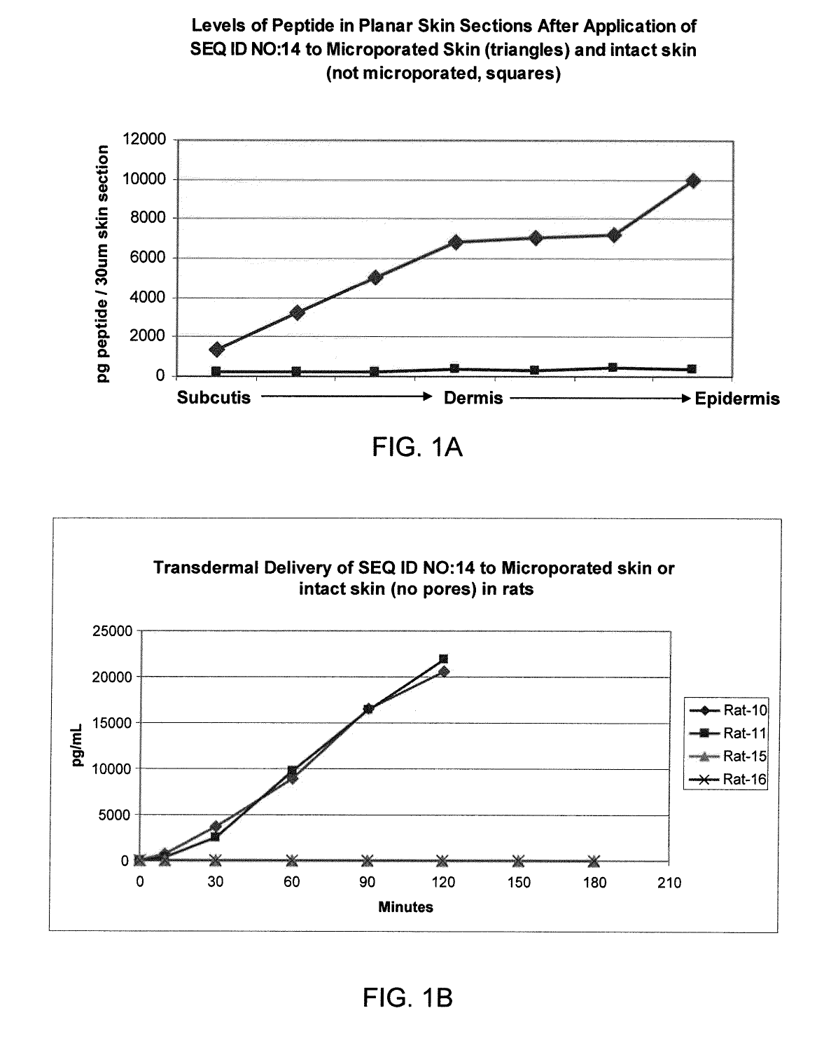

In the Examples set forth below, isozyme selective PKC modulatory peptides were administered transdermally to subjects in the form of linear conjugates of the isozyme selective peptide and a carrier peptide. For example, the εPKC inhibitor peptide EAVSLKPT (SEQ ID NO:13) attached to a TAT carrier peptide to form a conjugate was applied to microporated skin of animals and the blood level of the εPKC inhibitor peptide was analyzed as a function of time to ascertain uptake of the peptide across microporated skin as compared to intact skin. Results are shown in FIGS. 1A-1B, and as seen transdermal delivery of the inhibitory peptide conjugate (SEQ ID NO: 14) was significantly enhanced in the animals when the skin was microporated prior to application of the conjugate, relative to the animals wherein the conjugate (SEQ ID NO: 14) was applied to intact skin. FIG. 1B shows that the intact conjugate (SEQ ID NO:14) was present in the blood stream of the animals receiving the conjugate across microporated skin in an increasing amount with time.

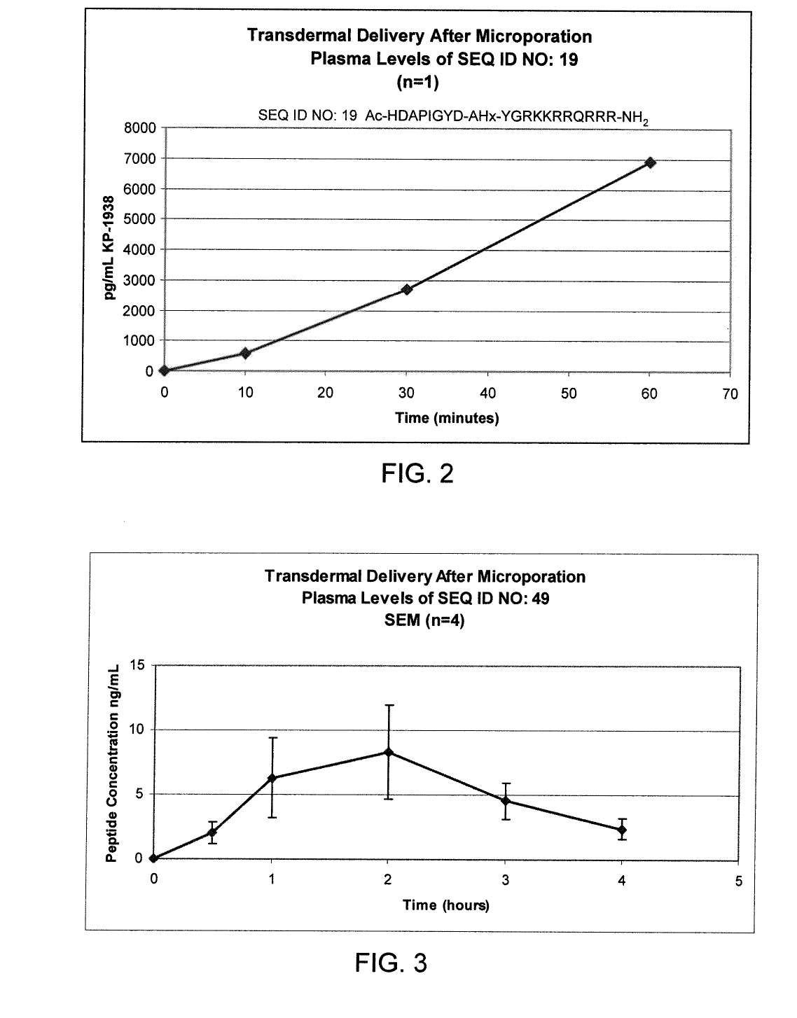

Example 2 and FIG. 2 show data illustrating that transdermal delivery of the εPKC inhibitor peptide HDAPIGYD (SEQ ID NO:18) attached to a TAT carrier peptide to form the conjugate identified as SEQ ID NO: 19 was enhanced in animals with microporated skin and the conjugate itself (SEQ ID NO:19) was present in the blood stream of the animals in an increasing amount with time.

Example 3 describes another study where the γV5 peptide RLVLAS (SEQ ID NO: 43) with an acyl (Ac) end cap (R—CO, where R is CH₃) was attached by a GG linker to a TAT carrier peptide (SEQ ID NO: 46) and then the C-terminus was amidated (NH₂) to form a conjugate identified as SEQ ID NO: 49. This conjugate was delivered transdermally to microporated skin, and the concentration of the conjugate in the blood measured as a function of time. The results are shown in FIG. 3.

These studies show that the full conjugate, i.e., the intact conjugate comprised of the PKC modulatory peptide linked to the carrier peptide, was successfully delivered through the microporated skin. The site of action of the isozyme selective PKC peptide is intracellular, so it is desirable that the carrier peptide remain attached to the PKC peptide upon transport through the layers of the skin. Prior to these studies, it was unknown and unpredictable whether such a conjugate delivered transdermally could or would enter and cross through the layers of skin beneath the stratum corneum, and/or be delivered to the systemic circulation. Based on these findings, a skilled artisan will appreciate that any peptide, not limited to PKC modulatory peptides, and any peptidomimetic when attached to a carrier peptide, preferably in the form of a linear fusion conjugate, optionally stabilized with terminal caps, can be administered through microporated skin to achieve delivery of the conjugate into systemic circulation and/or locally, wherein the conjugate is available for intracellular uptake.

### III. Methods of Treatment

From the foregoing discussion, a skilled artisan can appreciate the therapeutic effect depends on the PKC modulatory peptide in the conjugate that is applied transdermally. In one embodiment, the PKC modulatory peptide has isozyme selective activity to inhibit delta PKC for treatment or prevention of reperfusion injury. In another embodiment, the PKC modulatory peptide has isozyme selective activity to activate delta PKC for induction of apoptosis, for enhancing a chemotherapeutic regimen. In another embodiment, the PKC modulatory peptide has isozyme selective activity to inhibit alpha PKC for inhibition of metastases. In another embodiment, the PKC modulatory peptide has isozyme selective activity to inhibit a beta PKC for anti-angiogenesis and anti-proliferation, useful in cancer therapies. In another embodiment, the PKC modulatory peptide has isozyme selective activity to activate espilon PKC for induce protection of tissue from ischemic injury.

In the treatment methods, an effective amount of the PKC modulatory peptide is provided in the form of the conjugate. An “effective amount” comprises an amount that results in treatment of a condition or attenuation of a symptom of the condition. An effective amount will vary from subject to subject depending on the subject's normal sensitivity to pain, its height, weight, age, and health, the condition, the particular modulatory peptide administered, and other factors. As a result, it is advisable to empirically determine an effective amount for a particular subject under a particular set of circumstances.

In one embodiment, a PKC modulatory peptide is administered transdermally or intradermally in an amount sufficient for attenuation of pain. As used herein, attenuation of pain typically intends a lessening of pain, and in some embodiments can intend preventing future pain, and/or inhibiting heightened sensitivity to noxious or painful stimuli (hyperalgesia) or a painful response to a normally innocuous stimulus (allodynia). In one embodiment, pain is treated by delivering the PKC modulatory compound to a target tissue, by either systemic delivery or by localized delivery. The ability of the peptides to lessen pain, via selective inhibition of an isozyme of PKC, upon transdermal application to microporated skin reduces unwanted side effects. Peptide inhibitors of γPKC and/or εPKC, in one embodiment, reduce hyperalgesia without affecting nociception or compromising other sensory perception.

Pain is a basic clinical symptom seen by physicians and is often categorized as mild, moderate, or severe. The γPKC and/or εPKC modulatory peptides described herein are suitable for treatment of pain in any of these categories. For example, cancer and post-operative surgical pain are often described as being in the moderate-to-severe category. Tumor infiltration of bone, nerve, soft tissue, or viscera are common causes of cancer pain. Various factors influence the prevalence of cancer pain in patients, such as the tumor type, state, and site, as well as patent variables. With respect to post-operative pain, the severity of the pain is often dependent on location and extent of intervention.

More particularly, γPKC and/or εPKC modulatory peptides are suited to treatment of acute or chronic pain caused, for example, by neuropathic or inflammatory conditions. Exemplary inflammatory conditions contemplated for treatment include, but are not limited to, sunburn, osteoarthritis, colitis, carditis, dermatitis, myostis, neuritis, and rheumatoid arthritis, lupus and other collagen vascular diseases, as well as post-operative surgical pain. Conditions associated with neuropathic pain include, but are not limited to, trauma, surgery, amputation, abscess, demyelinating diseases, trigeminal neuralgia, cancer, chronic alcoholism, stroke, thalamic pain syndrome, diabetes, herpes infections, and the like.

Inflammation and nerve damage can induce hyperalgesia, where a noxious stimulus is perceived as intensely painful due to a lowering of pain threshold. Accordingly, in its embodiments addressed to the treatment of pain, described herein are a composition and a method for treating hyperalgesia in a patient. Additionally, described herein are compositions and methods for treating allodynia in a subject; that is, treating the pain associated with a normally non-noxious stimulus.

One embodiment is the treatment of a patient having inflammatory pain. Such inflammatory pain may be acute or chronic and can be due to any number of conditions characterized by inflammation including, without limitation, sunburn, rheumatoid arthritis, osteoarthritis, colitis, carditis, dermatitis, myositis, neuritis and collagen vascular diseases. Another embodiment is the treatment of a patient having neuropathic pain. Such patients can have a neuropathy classified as a radiculopathy, mononeuropathy, mononeuropathy multiplex, polyneuropathy or plexopathy. Diseases in these classes can be caused by a variety of nerve-damaging conditions or procedures, including, without limitation, trauma, stroke, demyelinating diseases, abscess, surgery, amputation, inflammatory diseases of the nerves, causalgia, diabetes, collagen vascular diseases, trigeminal neuralgia, rheumatoid arthritis, toxins, cancer (which can cause direct or remote (e.g. paraneoplastic) nerve damage), chronic alcoholism, herpes infection, AIDS, and chemotherapy. Nerve damage causing hyperalgesia can be in peripheral or CNS nerves.

The term “lessening pain” as used herein comprises a process by which the level of pain a subject perceives is reduced relative to the level of pain the subject would have perceived were it not for the intervention. Where the subject is a person, the level of pain the person perceives can be assessed by asking him or her to describe the pain or compare it to other painful experiences. Alternatively, pain levels can be calibrated by measuring the subject's physical responses to the pain, such as the release of stress-related factors or the activity of pain-transducing nerves in the peripheral nervous system or the CNS. One can also calibrate pain levels by measuring the amount of a well characterized analgesic required for a person to report that no pain is present or for a subject to stop exhibiting symptoms of pain. Lessening pain can result from increasing the threshold at which a subject experiences a given stimulus as painful. It can result from inhibiting hyperalgesia, the heightened sensitivity to a noxious stimulus, and such inhibition can occur without impairing nociception, the subject's normal sensitivity to a noxious stimulus. “A subject in need thereof” comprises an animal or person, expected to experience pain in the near future. Such animal or person may have a ongoing condition that is causing pain currently and is likely to continue to cause pain, or the animal or person has been, is or will be enduring a procedure or event that usually has painful consequences. Chronic painful conditions such as diabetic neuropathic hyperalgesia and collagen vascular diseases are examples of the first type; dental work, particularly in an area of inflammation or nerve damage, and toxin exposure (including exposure to chemotherapeutic agents) are examples of the latter type.

The difference between “acute” and “chronic” pain is one of timing: acute pain is experienced soon (e.g., within about 48 hours, about 24 hours, or about 12 hours) after the occurrence of the event (such as inflammation or nerve injury) that led to such pain. By contrast, there is a significant time lag between the experience of chronic pain and the occurrence of the event that led to such pain. Such time lag is at least about 48 hours after such event, e.g., at least about 96 hours after such event, or at least about one week after such event.

Neuropathic pain comprises pain arising from conditions or events that result in nerve damage. Neuropathy comprises a disease process resulting in damage to nerves. Causalgia denotes a state of chronic pain following nerve injury or a condition or event, such are cardiac infarction, that causes referred pain. Allodynia comprises a condition in which a person experiences pain in response to a normally nonpainful stimulus, such as a gentle touch. An analgesic agent comprises a molecule or combination of molecules that causes a reduction in pain.

Activity and potency of the εPKC and γPKC inhibitory peptides described above for modulating pain may be investigated using one or more models of pain or can be readily analyzed in simple in vivo studies, such as those described in the examples below. An exemplary model is an acute inflammatory pain induced by capsaicin or by formalin. This model, and others, having long-term increases of sensitivity to noxious stimuli can be useful in modeling certain human pathological pain. The capsaicin model of inflammation, together with a low rate thermal test, mimics central sensitization and hyperalgesia resulting from chronic pain. Application of capsaicin to the skin produces a robust, hours-long, C fiber selective hyperalgesia indicated by significant lowering of paw withdrawal latencies during low heating rate thermal tests. The receptor for capsaicin (VR-1 vanilloid receptor found on C fibers) has been recently cloned. It is a ligand-gated, non-selective cation channel. In addition to responding to capsaicin, VR-1 also responds to thermal stimuli (approximately 43° C.) (Kidd B. L., et al., Br. J. Anaesth., 87(1):3-11 (2001)) and to protons, suggesting that its activity is enhanced during inflammation. Capsaicin has been shown to selectively activate and sensitize C fibers, and not Aδ. Therefore, Aδ latency measurements are used as controls for animal wellbeing during the studies.

Another exemplary model is the formalin model in rodents, which has been validated as a predictive test of treating injury-induced pain in humans (Dennis, S. G. and Meizack, R., Advances in Pain Research and Therapy, Vol. 3, 747, Eds. J. J. Bonica et al., Raven Press, New York, 1979; Tjolsen, A., et al., Pain, 51:5-17 (1992)). The model produces a bi-phasic response, where the initial phase is triggered by a primary afferent barrage, similar in character to that described for the acute phasic tests except that chemical nociceptors are the mediators. The second phase is considered to be the hyperalgesic spontaneous activity that results from the initial tissue damage and reflects the lowering of nociceptive threshold plus the priming or “wind up” of the corresponding spinal circuitry. Thus, both peripheral and central neuronal circuits and mediators are required to induce and sustain this painful tissue-injury condition.

### EXAMPLES

The following examples are illustrative of the methods described herein, and are in no way intended to limit the methods.

### Example 1

### Transdermal Delivery of PKC Peptide Inhibitors to Micorporated Skin

Male CD Hairless rats were weighed (range: 300-500 grams) and anesthetized by inhaled isoflurane. The rats remained under anesthesia for the duration of the experiment and were sacrificed after the last blood draw. Rats were cannulated at a femoral or jugular vein for blood draws. A derma roller with 1.0 or 1.5 mm microneedles (Moohan Dr. Roller) was rolled across a 1 cm² area of skin on the dorsal trunk or waist by passing the roller over the skin 5 times with moderate pressure. Fifty μL of the cPKC inhibitor peptide EAVSLKPT (SEQ ID NO:13) attached to a TAT carrier peptide to form the conjugate peptide identified as SEQ ID NO: 14 at 1% to 10% (10 to 100 mg/mL) was applied to the microporated skin and occluded with a 1 cm² piece of saran wrap. Blood draws are taken over a 2-3 hour period. Blood was spun in a micro-centrifuge at 12,000 rpm for 2 minutes. Plasma was transferred to a new tube and stored frozen (−70° C.) until analysis. As a control, rats with intact skin (i.e., rats not subjected to the microneedle array) were also treated with the same peptide conjugate.

To analyze levels of the εPKC inhibitor peptide EAVSLKPT (SEQ ID NO:13) in plasma, both ELISA on planar skin sections and a sandwich ELISA that uses antibodies that recognize different portions of the conjugate (the εPKC inhibitor peptide and the TAT carrier peptide) were employed. The conjugate concentrations were calculated from a standard curve that is generated by 5 parameter curve fitting using StatLIA software. The limit of quantification of the ELISA was 30 pg/mL. Bioanalysis of other peptides tested were done using a similar method with appropriate antibodies.

Results are shown in FIGS. 1A-1B. Transdermal delivery of the inhibitory peptide conjugate (SEQ ID NO: 14) was significantly enhanced in the animals treated with microporation relative to the animals with the inhibitory peptide conjugate (SEQ ID NO: 14) applied to intact skin. Microporation increased penetration of the peptide conjugate through the stratum corneum and into the underlying skin (FIG. 1A) and with time (FIG. 1B).

### Example 2

### Transdermal Delivery of PKC Peptide Inhibitors to Micorporated Skin

Transdermal delivery of the εPKC inhibitor peptide HDAPIGYD (SEQ ID NO:18) attached to a TAT carrier peptide to form the conjugate peptide identified as SEQ ID NO: 19 was performed as described in Example 1. Results are shown in FIG. 2.

### Example 3

### Transdermal Delivery of PKC Peptide Inhibitors to Micorporated Skin

The γV5 peptide RLVLAS (SEQ ID NO: 43) with an acyl (Ac) end cap was attached by a GG linker to a TAT carrier peptide (SEQ ID NO: 46) and then the C-terminus was amidated (NH₂) to form a conjugate identified as SEQ ID NO: 49. Transdermal delivery of this conjugate was performed as described in Example 1 except that a 1 cm² solid polymer microneedle array was used to microporate the skin and an aqueous reservoir patch was used to contact a 10% aqueous formulation of the conjugate with the microporated skin. Results are shown in FIG. 3, where the amount of conjugate in the blood, in ng/mL, is shown as a function of time.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A method for delivery of an isozyme specific PKC modulatory peptide, comprising:
administering a therapeutically effective amount of a conjugate comprised of an isozyme specific PKC modulatory attached to a carrier peptide, wherein said administering comprises application of the conjugate to microporated skin, and wherein said administering achieves administration of the conjugate systemically.

2. The method of claim 1, wherein said administering comprises application of the conjugate to skin microporated prior to or simultaneous with application of said conjugate.

3. The method of claim 2, wherein said administering comprises application of the conjugate to skin microporated by a technique selected from a microneedle array applied to the skin, thermal ablation, laser ablation, ultrasound, or electroporation.

4. The method of claim 2, wherein said administering comprises application of a microneedle array to the skin, and wherein said conjugate is disposed on an interior or an exterior surface of microneedles in the microneedle array.

5. The method of claim 1, further comprising occluding the microporated skin after application of said conjugate.

6. The method of claim 1, wherein the carrier peptide is selected from the group consisting of Antennapedia homeodomain-derived carrier peptide, a Transactivating Regulatory Protein (Tat)-derived transport polypeptide from the Human Immunodeficiency Virus, and a polyarginine.

7. The method of claim 1, wherein the PKC modulatory peptide has a sequence that has 80% sequence identity with 6-20 contiguous amino acid residues from SEQ ID NO: 1 or SEQ ID NO: 2.

8. The method of claim 1, wherein the PKC modulatory peptide has a sequence that has 80% sequence identity with 6-20 contiguous amino acid residues from SEQ ID NO: 3 or SEQ ID NO: 4.

9. The method of claim 1, wherein the PKC modulatory peptide has a sequence with at least 80% sequence identity to SEQ ID NO: 13, SEQ ID NO: 18, SEQ ID NO: 43, or SEQ ID NO: 50.

10. The method of claim 1, wherein the conjugate has a sequence identified as SEQ ID NO: 14, SEQ ID NO: 11, SEQ ID NO: 49, or SEQ ID NO: 51.

11. The method of claim 1, wherein said PKC inhibitor peptide or said conjugate is modified to with a N-terminal or C-terminal chemical moiety.

12. A method, comprising:
contacting microporated skin with a therapeutic conjugate peptide, said conjugate peptide comprised of a PKC inhibitor peptide having isozyme selective activity for an isozyme of PKC attached to a carrier peptide.

13. The method of claim 12, wherein said contacting comprises microporating a region of skin followed by application of said conjugate peptide.

14. The method of claim 12, wherein said contacting comprises simultaneously microporating a region of skin and application of said conjugate peptide.

15. The method of claim 12, wherein said contacting comprises contacting skin microporated by a microneedle array applied to the skin, laser ablation, ultrasound, or electroporation.

16. The method of claim 12, wherein the carrier peptide is selected from the group consisting of Antennapedia homeodomain-derived carrier peptide, a Transactivating Regulatory Protein (Tat)-derived transport polypeptide from the Human Immunodeficiency Virus, and a polyarginine.

17. The method of claim 12, wherein the PKC modulatory peptide has a sequence that has 80% sequence identity with 6-20 contiguous amino acid residues from SEQ ID NO: 1 or SEQ ID NO: 2.

18. The method of claim 12, wherein the PKC modulatory peptide has a sequence that has 80% sequence identity with 6-20 contiguous amino acid residues from SEQ ID NO: 3 or SEQ ID NO: 4.

19. The method of claim 12, wherein said PKC modulatory peptide or said conjugate is modified with a N-terminal or C-terminal chemical moiety.

20. The method of claim 12, wherein the PKC modulatory peptide has a sequence with at least 80% sequence identity to SEQ ID NO: 13, SEQ ID NO: 18, SEQ ID NO: 43, or SEQ ID NO: 50.

21. The method of claim 12, wherein the conjugate has a sequence identified as SEQ ID NO: 14, SEQ ID NO: 11, SEQ ID NO: 49, or SEQ ID NO: 51.
